(19) United States
(12) Patent Application Publication
(10) Pub. No.: US 2024/0175082 A1
(43) Pub. Date: May 30, 2024

(54) MOLECULES COMPRISING BRANCHED LINKERS AND METHODS OF USE

(71) Applicant: 10x Genomics, Inc., Pleasanton, CA (US)

(72) Inventor: Justin COSTA, Union City, CA (US)

(21) Appl. No.: 18/488,502

(22) Filed: Oct. 17, 2023

Related U.S. Application Data
(60) Provisional application No. 63/416,900, filed on Oct. 17, 2022.

(51) Int. Cl. C12Q 1/6841; C12Q 1/682

(52) U.S. Cl. CPC C12Q 1/6841; C12Q 1/682

(57) ABSTRACT

The present disclosure in some aspects relates to methods and compositions for analyzing a biological sample comprising contacting the biological sample with a molecule comprising a branched linker. In some embodiments, the molecule is a detectable probe comprising branches each comprising one or more detectable labels and/or one or more sequences for hybridizing to a detectably labeled probe. In some embodiments, the molecule comprises an oligonucleotide linked to photoactivatable functional moieties via a branched linker.

### CROSS-REFERENCE TO RELATED APPLICATIONS

This application claims priority to U.S. Provisional Patent Application No. 63/416,900, filed Oct. 17, 2022, entitled “MOLECULES COMPRISING BRANCHED LINKERS AND METHODS OF USE,” which is herein incorporated by reference in its entirety for all purposes.

### FIELD

The present disclosure relates in some aspects to methods and compositions for analyzing a biological sample, including linkers (e.g., branched linkers) for immobilizing molecules in a biological sample and/or boosting signal-to-noise ratios detected in the biological sample during in situ analysis.

### BACKGROUND

Methods are available for analyzing molecules in a biological sample in situ, such as in a cell or tissue sample. Current methods for analyzing analytes in situ can have low sensitivity and specificity, have limited plexity, or be biased, time-consuming, labor-intensive, and/or error-prone. Improved methods for in situ analyte detection are needed. The present disclosure addresses these and other needs.

### BRIEF SUMMARY

Nucleic acid and non-nucleic acid analytes can be detected using methods that involve the detection of nucleic acid molecules. For instance, detectable probes can be used to detect nucleic acid analytes, nucleic acid probes, and products of the analytes or probes (e.g., nucleic acid concatemers, such as rolling circle amplification (RCA) products (RCPs)) in a biological sample (e.g., a cell or tissue sample). In some aspects, provided herein are i) molecules comprising branched structures for immobilizing analytes, probes targeting the analytes, or products of the analytes or probes in a biological sample, and/or ii) molecules comprising branched structures for boosting the signals associated with analytes in the biological sample during in situ analysis. For example, introducing RCA primers with photoactivatable functional moieties could lead to improved tethering of the RCPs and thus immobilization of the RCPs in the sample, which may allow for better resolution of signals, resolution of signals associated with different analytes, and/or improved spatial fidelity and detection during downstream analyses. Furthermore, detectable probes or complexes thereof comprising a plurality of detectable labels may give rise to increased signal intensity and signal-to-noise ratios during in situ detection (e.g., in situ sequencing or sequential probe hybridization in situ).

In some embodiments, provided herein is a molecule comprising a branched linker. In some embodiments, the molecule is a detectable probe comprising branches each comprising one or more detectable labels and/or one or more sequences for hybridizing to a detectably labeled probe. In some embodiments, the detectable probe comprises multiple fluorophores in the branches such that the detectable probe has a higher photon count and increased signal-to-noise ratio when used for detection, as compared to a detectable probe having one fluorophore per probe. In some embodiments, the molecule comprises an oligonucleotide linked to photoactivatable functional moieties via a branched linker. In some embodiments, the oligonucleotide or a product thereof can be immobilized to molecules in a sample via the photoactivatable functional moieties, thereby increasing the positional stability of the oligonucleotide or product thereof during in situ analysis.

In some embodiments, provided herein are methods and compositions for nucleic acid molecule analysis (e.g., detection) in a biological sample. In some embodiments, the present disclosure provides methods for analyzing structures comprising nucleic acid molecules, such as nucleic acid concatemers (e.g., RCPs), using branched linkers to allow the coupling of a plurality of functional moieties to an oligonucleotide probe for analyzing the structures comprising nucleic acid molecules in situ. In particular aspects, the branched linkers may comprise amidite (e.g., phosphoramidite). The functional moiety may be a photoactivatable moiety, such as a diazirine moiety, on an oligonucleotide such as an RCA primer that can promote the tethering of the oligonucleotide (e.g., the RCA primer and the RCP generated therefrom) to endogenous molecules in the biological sample. In some embodiments, one or more arms of a branched linker may be coupled (directly or indirectly, such as via a covalent bond/linker to via nucleic acid hybridization) to one or more detectable labels to generate a branched probe, such as a detectable probe comprises branches coupled to fluorescent moieties, to improve the signal intensity during detection and analysis of analytes in a sample.

In some aspects, disclosed herein is a method for analyzing a biological sample, comprising contacting the biological sample with a probe comprising: i) a hybridization region complementary to a target nucleic acid sequence, ii) a plurality of detectable labels, and iii) a branched linker coupled to the hybridization region, wherein the branched linker comprises a plurality of arms, wherein one or more of the arms are coupled to one or more detectable labels. In some embodiments, each of the plurality of arms is coupled to one or more detectable labels, e.g., fluorophores. In any of the embodiments herein, the biological sample can be a tissue sample such as a tissue section. In any of the embodiments herein, the probe may hybridize to a target molecule at a location in the biological sample, wherein the target molecule comprises one or more copies of the target nucleic acid sequence. In any of the embodiments herein, the method may comprise detecting signals associated with the plurality of detectable labels at the location in the biological sample. In any of the embodiments herein, one or more of the plurality of arms can comprise a spacer between detectable labels. In any of the embodiments herein, the plurality of arms and the plurality of detectable labels in the probe may be configured such that detectable labels in the same arm and/or detectable labels in different arms do not quench one another.

In any of the embodiments herein, the target molecule may be: i) a DNA concatemer, or ii) an intermediate probe that directly or indirectly binds to a DNA concatemer. In any of the embodiments herein, the DNA concatemer may be a rolling circle amplification (RCA) product (RCP). In any of the embodiments herein, the RCP may comprise a plurality of barcode sequences. In some embodiments, the RCP comprises a barcode sequence corresponding to an analyte or a portion thereof in the biological sample. In any of the embodiments herein, the RCP may be a product of a nucleic acid molecule in the biological sample or a product of a circular or circularizable probe or probe set that hybridizes to the nucleic acid molecule.

In any of the embodiments herein, the nucleic acid molecule may be genomic DNA, mRNA, or cDNA. In any of the embodiments herein, the RCP may be a product of a reporter oligonucleotide of a labelling agent that directly or indirectly binds to an analyte in the biological sample, or a product of a circular or circularizable probe or probe set that hybridizes to the reporter oligonucleotide. In any of the embodiments herein, the labelling agent may comprise a binding moiety that directly or indirectly binds to a nucleic acid analyte and/or a non-nucleic acid analyte in the biological sample.

In any of the embodiments herein, the RCP may be a nanoball having a diameter between about 0.05 μm and about 3 μm, between about 0.1 μm and about 0.5 μm, between about 0.1 μm and about 0.4 μm, between about 0.2 μm and about 0.3 μm, between about 0.3 μm and about 0.4 μm, or between about 0.5 μm and about 1 μm. In any of the embodiments herein, the RCP may be a nanoball having a diameter between about 0.05 μm and about 1 μm. In any of the embodiments herein, the RCP may be a nanoball having a diameter between about 0.1 μm and about 0.8 μm. In any of the embodiments herein, the RCP may be a nanoball having a diameter between about 0.3 μm and about 0.5 μm. In any of the embodiments herein, the RCP may be a nanoball having a diameter between about 0.1 μm and about 0.4 μm.

In any of the embodiments herein, the RCP may be between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length. In any of the embodiments herein, the RCP may be between about 45 and about 70 kilobases in length. In any of the embodiments herein, the RCP may comprise between about 10 and about 100, between about 100 and about 1,000, between about 1,000 and about 5,000, between about 5,000 and about 10,000, or more than 10,000 copies of a unit sequence corresponding to the rolling circle amplification template. In any of the embodiments herein, the RCP may be generated in situ.

In any of the embodiments herein, the biological sample may comprise a plurality of RCPs, and the method comprises detecting signals associated with the plurality of RCPs at locations in the biological sample. In some embodiments, about 90% or more of the plurality of RCPs in the biological sample have a diameter of about 500 nm or less. In any of the embodiments herein, about 90% or more of the plurality of RCPs in the biological sample may have diameters between about 100 nm and about 400 nm. In any of the embodiments herein, the median size of the plurality of RCPs may be about 500 nm or less. In any of the embodiments herein, the median size of the plurality of RCPs may be about 350 nm or less.

In any of the embodiments herein, the method may comprise generating the RCP(s) in situ in the biological sample. In any of the embodiments herein, the tissue sample may be a tissue slice between about 1 μm and about 50 μm in thickness. In any of the embodiments herein, the tissue sample may be between about 5 μm and about 35 μm in thickness. In some embodiments, the biological sample is not a metaphase spread or interphase cell sample.

In any of the embodiments herein, one or more of the plurality of arms of the branched linker can be directly coupled to one or more of the plurality of detectable labels. In any of the embodiments herein, one or more of the plurality of arms can be indirectly coupled to one or more of the plurality of detectable labels via a linker. In any of the embodiments herein, one or more of the plurality of arms may be covalently coupled to one or more of the plurality of detectable labels. In any of the embodiments herein, one or more of the plurality of arms may be noncovalently coupled to one or more of the plurality of detectable labels.

In any of the embodiments herein, each of the plurality of detectable labels may comprise a fluorescent moiety. In any of the embodiments herein, the probe may comprise at least about 3, at least about 5, at least about 10, at least about 15, at least about 20, or more than 20 molecules of the same detectable label or different detectable labels.

In any of the embodiments herein, the hybridization region of the probe that is complementary to the target nucleic acid sequence may be prepared synthetically. In some embodiments, the hybridization region complementary to the target nucleic acid sequence is prepared synthetically by coupling a nucleoside phosphoramidite to a growing oligomer strand in the 3′-5′ direction. In some embodiments, the DNA synthesis is carried out on a solid support (e.g., universal support).

In any of the embodiments herein, the branched linker may be directly coupled to the hybridization region. In any of the embodiments herein, the branched linker may be indirectly coupled to the hybridization region. In any of the embodiments herein, the branched linker may be covalently coupled to the hybridization region. In any of the embodiments herein, the branched linker may be noncovalently coupled to the hybridization region.

In any of the embodiments herein, the branched linker of the probe may comprise a symmetric double branch point linker, an asymmetric double branch point linker, a triple branch point linker, and/or a linker with more than three branch points.

In any of the embodiments herein, the branched linker of the probe may be coupled to the 3′ or 5′-end of the hybridization region. In any of the embodiments herein, the probe may comprise two or more branched linkers. In some embodiments, the probe is not a dendrimer. In any of the embodiments herein, the probe may comprise a first branched linker coupled to the 3′ of the hybridization region and a second branched linker coupled to the 5′ of the hybridization region. In any of the embodiments herein, the hybridization region may comprise an oligonucleotide or analog thereof. In any of the embodiments herein, the hybridization region may comprise a DNA oligonucleotide and/or a morpholino.

In any of the embodiments herein, the branched linker of the probe may be covalently coupled to the hybridization region complementary to the target nucleic acid sequence. In some embodiments, the branched linker is covalently bonded to the 5′-end of the hybridization region complementary to the target nucleic acid sequence. In some embodiments, the branched moiety is generated via a reaction between 5′-nucleoside of the hybridization region complementary to the target nucleic acid sequence and a branching reagent. In some such embodiments, the branching reagent is a phosphoramidite molecule comprising two or more arms. In some embodiments, the branching agent includes two arms. In some embodiments, the branching agent includes three arms. In some embodiments, the branching agent is a long trebler phosphoramidite. In some embodiments, the hybridization region complementary to the target nucleic acid sequence comprises a DNA oligonucleotide and/or a morpholino.

In any of the embodiments herein, each of the arms of the branching agent (e.g., long trebler phosphoramidite) of the probe is protected with a protecting group. In some embodiments, the protecting group is a 4,4′-dimethoxytrityl (DMT) group. In some embodiments, the protecting group is a fluorenylmethoxycarbonyl (Fmoc) group. In some embodiments, the protecting group (e.g., DMT) is removed prior to reaction with a spacer, as set forth herein. In some embodiments, the protecting group (e.g., DMT group or Fmoc group) is removed with a weak or mild acid or base prior to reaction with the spacer, detectable label or agent comprising a photoactivatable functional moiety. In some embodiments, the protecting group (e.g., DMT group) is removed with dichloroacetic acid. In some embodiments, the protecting group (e.g., DMT group) is removed with trichloroacetic acid. In some embodiments, the protecting group (e.g., FMOC group) is removed with a base such as piperidine.

In any of the embodiments herein, the linker of the probe may comprise or be generated using any one or more of:

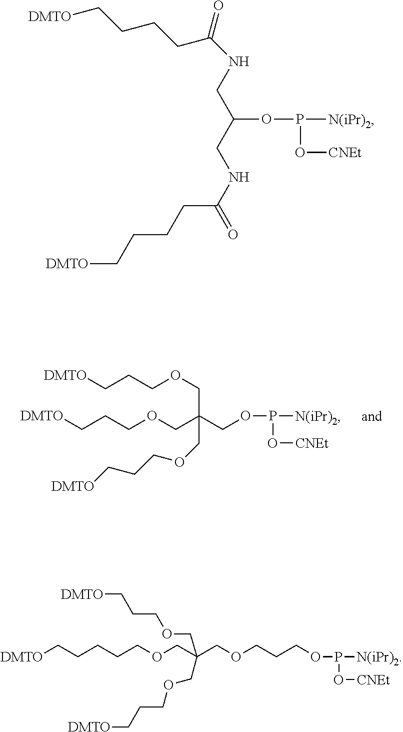

In any of the embodiments herein, the branched linker of the probe may comprise or be generated using any one or more of:

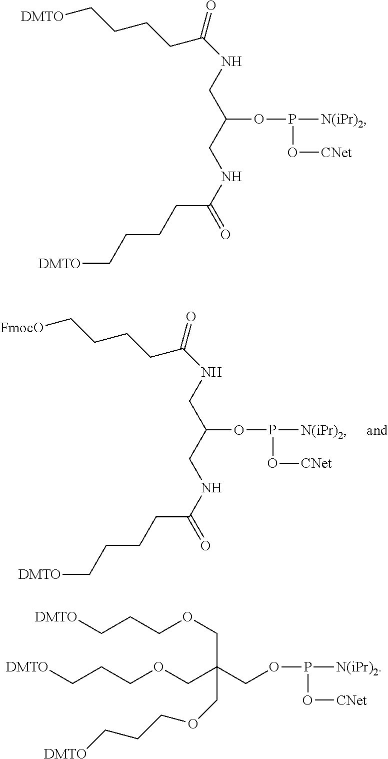

In embodiments with branched phosphoramidite branched linkers, such as those depicted in the preceding paragraph, the N(iPr)₂ group of the phosphoramidite may be cleaved following nucleophilic attack of the nucleoside at the 5′-end of the hybridization region of the probe. In some embodiments, the reaction between the hybridization region and the phosphoramidite is catalyzed by tetrazole.

In one embodiment, following reaction between the nucleoside at the 5′-end of the hybridization region and

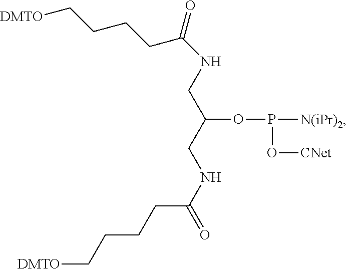

the resultant branched linker (prior to removal of the DMT protecting groups and the —OCNEt group) has the structure depicted below:

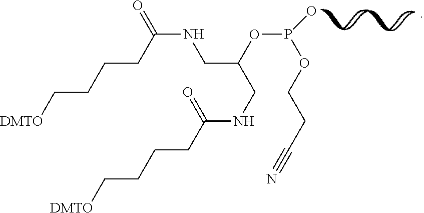

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate a compound having the structure depicted below:

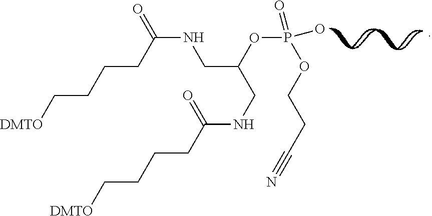

In some embodiments, oxidation is conducted with iodine (I₂), which increases the valency of the phosphorous from 3 to 5. Following removal of the DMT groups, the molecule depicted above can be added directly to a spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In some embodiments, the branched linker (prior to removal of the DMT protecting groups and the —OCNEt group) has the structure depicted below:

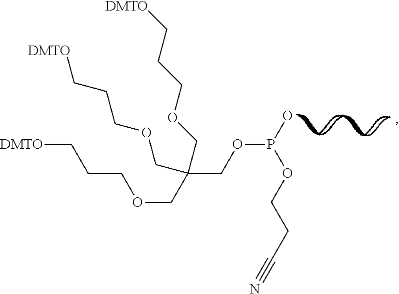

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate a compound has the structure depicted below:

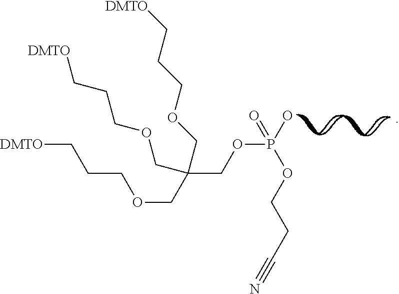

In some embodiments, oxidation is conducted with iodine (I₂), which increases the valency of the phosphorous from 3 to 5. Following removal of the DMT groups, the molecule depicted above can be added directly to a spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In some embodiments, the branched linker (prior to removal of the DMT protecting groups and the —OCNEt group) has the structure depicted below:

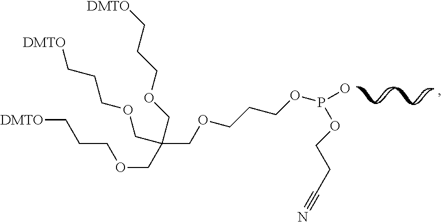

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate the compound depicted below:

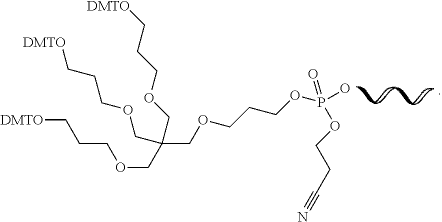

In some embodiments, oxidation is conducted with iodine (I₂), which increases the valency of the phosphorous from 3 to 5. Following removal of the DMT groups, the molecule depicted above can be added directly to spacer, detectable label or agent comprising a photoactivatable functional moiety, thereby generating the probe. In some embodiments, the —OCNEt group is cleaved prior to or following covalent attachment of the spacer, detectable label or agent comprising the photoactivatable functional moiety.

In any of the embodiments herein, the branched linker may comprise one or more non-nucleic acid moieties and/or one or more nucleic acid moieties. In any of the embodiments herein, the one or more nucleic acid moieties may comprise a homopolymeric sequence. In any of the embodiments herein, the homopolymeric sequence may be a poly(T) sequence.

In any of the embodiments herein, the branched linker may comprise one or multiple levels of branching. In any of the embodiments herein, the branched linker may comprise one or multiple levels of branching. In any of the embodiments herein, the branched linker may comprise: a first level branch moiety comprising n branches, and a second level branch moiety comprising m branches, wherein n and m are integers independent of each other, n is 2 or greater, and m is 0 or greater. In some embodiments, each of the n branches in the first level branching comprises m branches in the second level branching. In any of the embodiments herein, n and m may be independently 2, 3, or greater. In any of the embodiments herein, the first level branch moiety and/or the second level branch moiety may comprise an amidite. In any of the embodiments herein, the first level branch moiety and/or the second level branch moiety may comprise a phosphoramidite. In any of the embodiments herein, the branched linker may comprise one or more spacers in the first level branch moiety, in the second level branch moiety, and/or between the first level branch moiety and the second level branch moiety. In any of the embodiments herein, the one or more spacers may comprise an amidite. In any of the embodiments herein, the one or more spacers may comprise a phosphoramidite.

In any of the embodiments herein, the branched linker of the probe can comprise a flexible spacer molecule. In some embodiments, the flexible spacer is between one or more arms of the branched linker and the detectable label or the agent comprising a photoactivatable functional moiety. In some embodiments, the spacer provides a long hydrophilic attachment between one or more arms of the branched linker and the detectable label or agent comprising the photoactivatable functional moiety. In some embodiments, the spacer is a tetraethelyene glycol (TEG) spacer. In some embodiments, the spacer is a hexaetheylene glycol (HEG) spacer. In some embodiments, the spacer is a C6, C12, or C18 spacer. In some embodiments, the spacer is an 18-atom-hexa-ethtleneglycol spacer. In some embodiments, the spacers are added to one or more arms of the branched linkers following removal of the protecting groups (e.g., DMT groups) of the branched linkers. In some embodiments, the spacers can be covalently attached to the detectable labels or agent comprising the photoactivatable functional moiety.

In one embodiment, the probe has the structure depicted below:

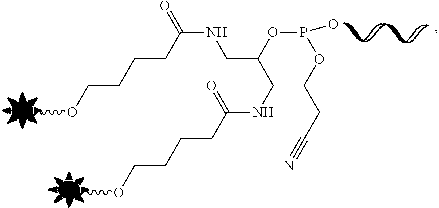

wherein [symbol] represents the hybridization region complementary to the target nucleic acid, [symbol] represents the spacer and [symbol] represents the detectable labels or agent comprising the photoactivatable functional moiety.

In one embodiment, the probe has the structure depicted below:

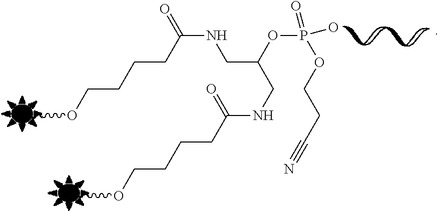

In one embodiment, the probe has the structure depicted below:

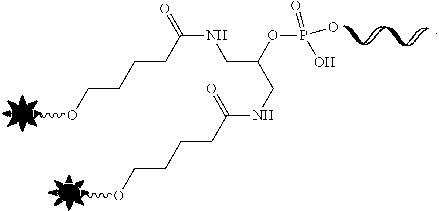

In one embodiment, the probe has the structure depicted below:

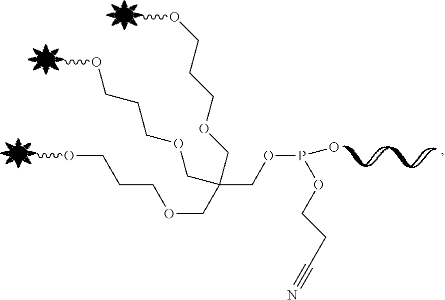

wherein [symbol] represents the hybridization region complementary to the target nucleic acid, [symbol] represents the spacer and [symbol] represents the detectable labels or agent comprising the photoactivatable functional moiety.

In one embodiment, the probe has the structure depicted below:

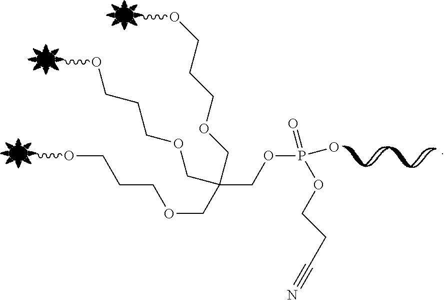

In one embodiment, the probe has the structure depicted below:

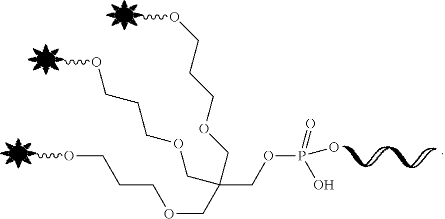

In one embodiment, the probe has the structure depicted below:

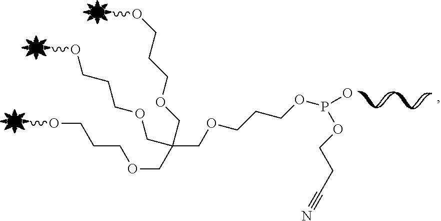

wherein [symbol] represents the hybridization region complementary to the target nucleic acid, [symbol] represents the spacer and [symbol] represents the detectable labels or agent comprising the photoactivatable functional moiety.

In one embodiment, the probe has the structure depicted below:

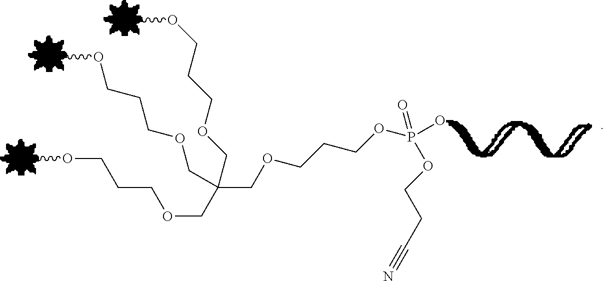

In one embodiment, the probe has the structure depicted below:

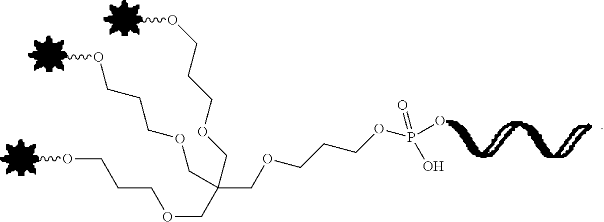

In some embodiments, the probe does not comprise a spacer between the detectable label or agent comprising the photoactivatable functional moiety and the arms of the branched linker. In such embodiments, the detectable label or agent comprising the photoactivatable functional moiety may be covalently attached directly to the arms of the branched linkers.

In any of the embodiments herein, the branched linker may comprise: a first level branch moiety comprising n branches, wherein n is an integer of 2 or greater. In some embodiments, the branched linker may comprise: a second level branch moiety comprising m branches, wherein m is an integer of 2 or greater. In any of the embodiments herein, the branched linker may comprise: a first level branch moiety comprising n branches and a second level branch moiety comprising m branches, wherein n and m are integers independently of 2 or greater. In any of the embodiments herein, the branched linker may comprise n×m branches each coupled to one or more fluorescent moieties, such as one or more molecules of the same fluorescent moiety and/or one or more molecules of different fluorescent moieties. In any of the embodiments herein, the 3′ and/or 5′ of the hybridization region may be coupled to the branched linker. In any of the embodiments herein, the 3′ or 5′ of the hybridization region may be coupled to one or more fluorescent moieties, e.g., without using a branched linker. In any of the embodiments herein, the probe may comprise at least about 3, at least about 5, at least about 10, at least about 15, at least about 20, or more than 20 molecules of the same detectable label. In any of the embodiments herein, the 3′ end of the hybridization region may be coupled to one or more fluorescent moieties.

In some aspects, provided herein is a method for analyzing a biological sample, comprising: a) contacting the biological sample with a circular probe or circularizable probe or probe set that hybridizes to a target analyte in the biological sample; b) generating a rolling circle amplification (RCA) product (RCP) in situ in the biological sample using the circular probe or a circularized probe generated from the circularizable probe or probe set as a template; c) contacting the biological sample with a hybridization probe comprising: i) a hybridization region complementary to a target nucleic acid sequence in the RCP or in an intermediate probe that directly or indirectly binds to the RCP, ii) one or more detectable labels, and iii) a linker coupled to the hybridization region, wherein the linker comprises one or more arms, and wherein the one or more arms are coupled to one or more detectable labels, thereby hybridizing the probe to the RCP or the intermediate probe in the biological sample; and d) detecting signals associated with the one or more detectable labels to detect the target analyte in the biological sample.

In some embodiments, provided herein is a method for analyzing a biological sample, comprising: a) contacting the biological sample with a circular probe or circularizable probe or probe set that hybridizes to a target analyte in the biological sample; b) generating a rolling circle amplification (RCA) product (RCP) in situ in the biological sample using the circular probe or a circularized probe generated from the circularizable probe or probe set as a template; c) contacting the biological sample with a hybridization probe comprising: i) a hybridization region complementary to a target nucleic acid sequence in the RCP or in an intermediate probe that directly or indirectly binds to the RCP, ii) a plurality of detectable labels, and iii) a branched linker coupled to the hybridization region, wherein the branched linker comprises a plurality of arms, and one or more of the arms are coupled to one or more detectable labels, thereby hybridizing the probe to the RCP or the intermediate probe in the biological sample; and d) detecting signals associated with the plurality of detectable labels to detect the target analyte in the biological sample.

In any of the embodiments herein, the RCP may comprise a plurality of the target nucleic acid sequence. In any of the embodiments herein, the target nucleic acid sequence may be a barcode sequence corresponding to the target analyte or a portion thereof. In any of the embodiments herein, the target nucleic acid sequence may be in the intermediate probe, and the RCP may comprise a plurality of a binding sequence for the intermediate probe. In any of the embodiments herein, upon hybridization of the intermediate probe to the binding sequence in the RCP, the intermediate probe may comprise a 3′ and/or a 5′ overhang, and the target nucleic acid sequence may be in the 3′ overhang and/or the 5′ overhang.

In any of the embodiments herein, the rolling circle amplification may be performed in situ in the biological sample for no more than 3 hours, no more than 2 hours, no more than 1 hour, no more than 30 minutes, or no more than 15 minutes.

In some aspects, provided herein is a method for analyzing a biological sample, comprising: a) contacting the biological sample with a circular probe or circularizable probe or probe set that hybridizes to a target analyte in the biological sample; b) generating a rolling circle amplification (RCA) product (RCP) in situ in the biological sample using the circular probe or a circularized probe generated from the circularizable probe or probe set as a template; c) contacting the biological sample with an oligonucleotide comprising: i) a hybridization region complementary to a target nucleic acid sequence in the RCP or in an intermediate probe that directly or indirectly binds to the RCP, ii) one or more photoactivatable functional moieties, and iii) a linker coupled to the hybridization region, wherein the linker comprises one or more arms, and wherein the one or more arms are coupled to one or more photoactivatable functional moieties, thereby hybridizing the oligonucleotide to the RCP or the intermediate probe in the biological sample; d) activating the one or more functional moieties by exposing the biological sample to UV light, wherein following exposure to the UV light, the one or more functional moieties react with one or more endogenous molecules in the biological sample, thereby immobilizing the RCP in the biological sample; and e) detecting the target analyte in the biological sample. In some embodiments, detecting the target analyte comprises contacting the biological sample with a detectable probe that directly or indirectly binds to the oligonucleotide or a product thereof.

In any of the embodiments herein referring to an analyte or product thereof, the product thereof can be an extension, amplification, and/or ligation product of the analyte. In some embodiments, the product thereof is a cDNA product of an RNA analyte.

In some embodiments, the rolling circle amplification may be primed using a primer comprising a functional moiety capable of covalently bonding to an endogenous molecule in the biological sample. In some embodiments, the endogenous molecule may be a protein or a nucleic acid, such as an RNA analyte. In any of the embodiments herein, the functional moiety may be photoactivatable. In any of the embodiments herein, the functional moiety may comprise a diazirine moiety, an aryl azide moiety, a benzophenone moiety, or an anthraquinone moiety. In any of the embodiments herein, the functional moiety may comprise a carbene forming moiety. In any of the embodiments herein, the carbene forming functional moiety may crosslink to a nucleic acid (e.g., an RNA analyte such as mRNA) at a random location in addition to a nearby endogenous molecule such as a protein.

In some embodiments, the oligonucleotide in step c) is prepared by reacting functional moieties on one or more of the arms of the linker bound to the hybridization domain with an agent comprising a photoactivatable functional moiety such as a diazirine moiety, an aryl azide moiety, a benzophenone moiety, or an anthraquinone moiety. In some embodiments, the linker covalently bonded to photoactivatable functional is a linker with a single arm (i.e., not branched). In some embodiments, the hybridization probe is generated via a reaction between 5′-nucleoside of the hybridization region complementary to the target nucleic acid sequence and a single-armed linker. In some embodiments, the single-armed linker is a phosphoramidite compound. As set forth with respect to the branched phosphoramidite linkers, following reaction between the 5′-nucleoside of the hybridization region and the single-armed phosphoramidite linker, the protecting group (e.g., DMT group) of the phosphoramidite linker can be chemically removed and the resultant unprotected phosphoramidite can be reacted directly with an agent comprising the photoactivatable functional moiety or with a spacer. In some embodiments, following covalent coupling of the phosphoramidite and the 5′-nucleoside of the hybridization region, oxidation is conducted with iodine (I₂), which increases the valency of the phosphorous on the phosphoramidite from 3 to 5.

In one embodiment, the single-armed linker used in the coupling reaction is 9-O-Dimethoxytrityl-triethylene glycol, 1-[(2-cyanoethyl)-(N,N-diisopropyl)]-phosphoramidite, also referred to as spacer phosphoramidite 9, which has the structure depicted below.

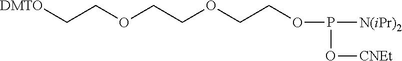

In another embodiment, the single-armed linker used in the coupling reaction is 18-O-Dimethoxytritylhexaethyleneglycol, 1-[(2-cyanoethyl)-(N,N-diisopropyl)]-phosphoramidite, also referred to as spacer phosphoramidite 9, which has the structure depicted below:

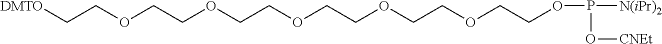

In another embodiment, the single-armed linker used in the coupling reaction is 12-(4,4′-Dimethoxytrityloxy)dodecyl-1-[(2-cyanoethyl)-(N,N-diisopropyl)]-phosphoramidite, also referred to as spacer C12 CE phosphoramidite, which has the structure depicted below:

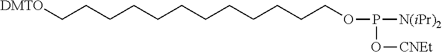

In another embodiment, the single-armed linker used in the coupling reaction is 3-(4,4′-Dimethoxytrityloxy)propyl-1-[(2-cyanoethyl)-(N,N-diisopropyl)]-phosphoramidite, also referred to as spacer phosphoramidite C3, which has the structure depicted below:

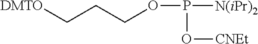

In some embodiments, the linker used in the coupling reaction is a branched linker with multiple arms, as described herein. In some such embodiments, the branched linker with multiple arms is a branched phosphoramidite linker.

In one embodiment where the oligonucleotide comprises a branched linker, all of the protecting groups (e.g., DMT or FMOC) are removed from the arms of branched linker prior to reaction with the agent comprising the photoactivatable functional moiety. The deprotected arms of the linker can then be reacted with the agent comprising a photoactivatable functional moiety, hence generating the photoactivatable probe. Alternatively, as set forth herein, one or more spacers can be added to the terminal ends of the arms of branched linkers following deprotection of the branched linkers. In such embodiments, the spacers may contain a functional moiety capable of reacting with the agent comprising the photoactivatable functional moiety. Reaction between the functional moiety on the spacer with the agent comprising a photoactivatable functional moiety generates the probe.

In some embodiments where the oligonucleotide comprises a branched linker, not all of the arms of the branched linker will include the photoactivatable functional moiety. For instance, in some embodiments where the branched linker includes two or three branches, only one of the arms will include the photoactivatable functional moiety. In these embodiments, the probes can be generated by selectively removing one of the protecting groups of the branched linker prior to reaction with the agent comprising the photoactivatable functional moiety. For instance, in a branched linker that includes two arms, where one arm is protected by DMT and the other arm is protected by FMOC, the FMOC can first be selectively removed and the DMT can be left intact. The resultant compound can then be added directly to the agent comprising the photoactivatable functional moiety or to a spacer. Following attachment of the agent comprising the photoactivatable functional moiety to the first arm, the DMT group of the second branch can be removed, hence generating the photoactivatable probe.

In some embodiments, the agent comprising the photoactivatable functional moiety is a diazirine moiety. In some such embodiments, the agent comprising the photoactivatable functional moiety is succinimidyl 4,4′-axipentanoate (NHS-Diazirine), sulfo-NHS-Diazirine, sulfosuccinimidyl 4,4′-azipentanoate (Sulfo-NHS-Diazirine), succinimidyl 6-(4,4′-azipentanamido)hexanoate (NHS-LC-Diazirine), succinimidyl 2-((4,4′-azipentanamido)ethyl)-1,3′-dithiopropionate (NHS-SS-Diazirine), or sulfo-NHS-SS-diazirine.

In any of the embodiments herein, the primer may comprise a primer region that hybridizes to the circular or circularized probe and a linker coupling the functional moiety to the primer region. In some embodiments, the linker comprises a branched structure comprising a plurality of arms each coupled to one or more copies of the functional moiety. In any of the embodiments herein, the linker may comprise an alkene. In any of the embodiments herein, the linker may comprise one or more bonds (e.g., ene bonds) and the linker is configured to enforce linearity of the linker away from the target analyte, the circular or circularizable probe, or the RCP. In any of the embodiments herein, the primer may comprise at least about 3, at least about 5, at least about 10, at least about 15, at least about 20, or more than 20 copies of the functional moiety.

In any of the embodiments herein, the method may comprise reacting the functional moiety with the endogenous molecule prior to, during, or after generating the RCP in the biological sample, thereby immobilizing the RCP in the biological sample. In some embodiments, the functional moiety is activated by exposing the biological sample to UV light, and the activated functional moiety reacts with the endogenous molecule. In any of the embodiments herein, the endogenous molecule may be a peptide or protein and the activated functional moiety may react with an amino acid side chain or a peptide backbone of the peptide or protein.

In some aspects, provided herein is a method for analyzing a biological sample, comprising: a) contacting the biological sample with an oligonucleotide comprising: i) a hybridization region complementary to a target nucleic acid sequence in a target molecule, ii) a photoactivatable functional moiety capable of covalently bonding to an endogenous molecule in the biological sample, and iii) a linker coupling the photoactivatable functional moiety to the hybridization region, thereby hybridizing the oligonucleotide to the target molecule at a location in the biological sample; b) photoactivating the photoactivatable functional moiety to react with the endogenous molecule, thereby immobilizing the oligonucleotide in the biological sample; c) contacting the biological sample with a detectable probe that directly or indirectly binds to the oligonucleotide or a product thereof, and d) detecting a signal associated with the detectable probe at the location in the biological sample. In some embodiments, the linker is a branched linker.

In any of the embodiments herein, the target molecule may be a genomic DNA, mRNA, or cDNA, or a product thereof. In any of the embodiments herein, the target molecule may be a nucleic acid probe that hybridizes to a genomic DNA, mRNA, or cDNA. In any of the embodiments herein, the target molecule may be a nucleic acid probe that hybridizes to a genomic DNA, mRNA, or cDNA.

In any of the embodiments herein, the target molecule may be a circular or circularizable probe or probe set, the oligonucleotide primes rolling circle amplification of the circular probe or a circularized probe generated from the circularizable probe or probe set, and the product of the oligonucleotide is a rolling circle amplification product (RCP).

In any of the embodiments herein, the endogenous molecule may be a protein, a nucleic acid, a carbohydrate, or a lipid. In any of the embodiments herein, the photoactivatable functional moiety may be a diazirine moiety, an aryl azide moiety, a benzophenone moiety, or an anthraquinone moiety. In any of the embodiments herein, the photoactivatable functional moiety may be a carbene forming moiety.

In any of the embodiments herein, the linker may comprise a plurality of arms each coupled to one or more copies of the photoactivatable functional moiety. In any of the embodiments herein, the linker may comprise an alkene. In any of the embodiments herein, the linker may comprise a double branch point linker, a triple branch point linker, and/or a linker with more than three branch points. In any of the embodiments herein, the linker may comprise an amidite. In any of the embodiments herein, the linker may comprise a phosphoramidite. In any of the embodiments herein, the oligonucleotide may comprise at least about 3, at least about 5, at least about 10, at least about 15, at least about 20, or more than 20 copies of the functional moiety. In any of the embodiments herein, the method may comprise photoactivating the photoactivatable functional moiety to react with the endogenous molecule prior to, during, or after generating the product of the oligonucleotide in the biological sample, thereby immobilizing the product in the biological sample. In any of the embodiments herein, the photoactivatable functional moiety may be photoactivated by exposing the biological sample to UV light, and the activated photoactivatable functional moiety may react with the endogenous molecule.

### BRIEF DESCRIPTION OF THE DRAWINGS

The drawings illustrate certain embodiments of the features and advantages of this disclosure. These embodiments are not intended to limit the scope of the appended claims in any manner.

FIGS. 1A-1C show exemplary detectable probes each comprising a branched linker with a plurality of arms. One or more of the arms are coupled to a detectable label (e.g., fluorescent moieties). Each detectable probe can comprise a hybridization region complementary to a target nucleic acid sequence.

FIG. 2 shows an exemplary molecule comprising a branched linker with a plurality of arms. One or more of the arms are coupled to a photoactivatable functional moiety (e.g., diazirine) capable of coupling to a nearby molecule (e.g., an endogenous protein) and/or a target analyte in a biological sample. The molecule can be used as a primer for RCA of a circular or circularized probe targeting the target analyte, and the RCA product generated in the biological sample can be immobilized to the nearby molecule and/or the target analyte by the branched molecule.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

When utilizing in situ analysis, such as rolling circle amplification (RCA), to detect nucleic acid targets, properly resolving densely packed nucleic acid concatemers, such as rolling circle amplification products (RCPs), in a cell or surface depends upon resolution of the individual nucleic acid concatemers. RCPs are nucleic acid concatemers comprising multiple copies of the target sequence and the RCA probe sequence, which can be visualized using in situ detection probes. Nucleic acid concatemer detection may be enhanced by immobilizing nucleic acid concatemers in the biological sample to prevent diffusion of the amplified concatemers, as restriction to a particular location in the sample results in stabilization and resolution of individual concatemers. Furthermore, detectably labeled probes (e.g., detection oligonucleotides comprising fluorescent labels) similarly present an opportunity for optimization of in situ analysis, as they are usually the last reagent contacted with the biological sample prior to imaging, and therefore have a low risk of affecting or requiring changes in upstream aspects involved with in situ detection.

Certain strategies for nucleic acid molecule immobilization and/or detection rely on the use of moieties i) for immobilization of the nucleic acid molecule, and/or ii) to increase signal brightness. However, some techniques only allow for a single functional moiety or detectable label to be conjugated to each oligonucleotide probe. One solution may be to utilize oligonucleotide probes that are significantly longer and contain multiple functional moieties and/or detectable labels, but this may have negative implications on the diffusional characteristics of the probes into thicker tissue sections. Moreover, due to length and specificity considerations for in situ analyses, longer probes may offer only a limited number of additional sites for the attachment of functional moieties and/or detectable labels. Thus, there is a need for methods and compositions allowing for one or more functional moieties and/or detectable labels on an individual probe without increasing the length of the probe.

Provided herein is a method for analyzing a biological sample (e.g., a cell or tissue sample), comprising contacting the biological sample with an oligonucleotide and/or probe comprising: i) a hybridization region complementary to a target nucleic acid sequence, ii) a plurality of detectable labels or photoactivatable functional moieties, and iii) a linker coupled to the hybridization region. The linker can be, in some embodiments, a branched linker, optionally comprising a plurality of arms. Branched linkers of the present invention may comprise an amidite moiety, for example, at the branch point of the arms. The amidite synthesis method allows for incorporation of various different combinations of chemicals (e.g., oligonucleotides of arbitrary sequence, one or more spacer moieties, double and triple branch points, etc). In contrast, traditional dendrimers tend to be composed of the same branching compounds iterated layer after layer. Purification is also more straightforward for the amidite synthesis approach compared to standard dendrimers thus ensuring increased homogeneity. Moreover, the amidite synthetic approach only involves one round of oligonucleotide/probe synthesis, while the dendrimer approach requires several rounds of synthesis and different oligonucleotide attachment chemistries. In some aspects, the dendrimer approach generates branch points equally in all directions from a center point, whereas the branched linkers and generated molecules provided herein have more polarity from the initiation point. The one or more of arms of the branched linker can be coupled to one or more detectable labels to allow for the hybridization of the probe to a target molecule, such as a RCP, at a location in the biological sample. Signals associated with the plurality of detectable labels at the location in the biological sample may be detected, thus detecting the target nucleic acid sequence in the biological sample. In particular aspects, the one or more arms of the branched linker are configured such that detectable labels in the same probe do not quench one another during the detecting.

In some aspects, provided herein is a method for analyzing a biological sample, comprising: a) contacting the biological sample with a probe comprising: i) a hybridization region complementary to a target nucleic acid sequence, ii) a plurality of detectable labels, and iii) a branched linker coupled to the hybridization region, wherein the branched linker comprises a plurality of arms, wherein one or more of the arms are coupled to one or more detectable labels, thereby hybridizing the probe to a target molecule at a location in the biological sample, wherein the target molecule comprises one or more copies of the target nucleic acid sequence, wherein the biological sample is a cell or a tissue sample; b) detecting signals associated with the plurality of detectable labels at the location in the biological sample.

In some aspects, provided herein is a method for analyzing a biological sample, comprising: a) contacting the biological sample with a circular probe or circularizable probe or probe set that hybridizes to a target analyte in the biological sample; b) generating a rolling circle amplification product (RCP) in situ in the biological sample using the circular probe or a circularized probe generated from the circularizable probe or probe set as a template; c) contacting the biological sample with a probe comprising: i) a hybridization region complementary to a target nucleic acid sequence in the RCP or in an intermediate probe that hybridizes to the RCP, ii) a plurality of detectable labels, and iii) a branched linker coupled to the hybridization region, wherein the branched linker comprises a plurality of arms each coupled to one or more detectable labels, thereby hybridizing the probe to the RCP or the intermediate probe in the biological sample; and d) detecting signals associated with the plurality of detectable labels to detect the target analyte in the biological sample. In some embodiments, the RCA is primed using a primer comprising a photoactivatable functional moiety capable of covalently bonding to an endogenous molecule in the biological sample. The photoactivatable functional moiety may comprise a carbon bound to two nitrogen atoms, which are double-bonded to each other, forming a cyclopropene-like ring, 3H-diazirene. The photoactivatable functional moiety may be a diazirine moiety, an aryl azide moiety, a benzophenone moiety, or an anthraquinone moiety (e.g., a carbene forming moiety).

In some aspects, provided herein is a method for analyzing a biological sample, comprising: a) contacting the biological sample with an oligonucleotide comprising: i) a hybridization region complementary to a target nucleic acid sequence in a target molecule, ii) a functional moiety capable of covalently bonding to an endogenous molecule in the biological sample, wherein the functional moiety is photoactivatable, and, iii) a linker coupling the functional moiety to the hybridization region, thereby hybridizing the oligonucleotide to the target molecule at a location in the biological sample; b) reacting the functional moiety with the endogenous molecule, thereby immobilizing the oligonucleotide in the biological sample; c) contacting the biological sample with a detectable probe that directly or indirectly binds to the oligonucleotide or a product thereof; and d) detecting a signal associated with the detectable probe at the location in the biological sample. The photoactivatable functional moiety may be a diazirine moiety, an aryl azide moiety, a benzophenone moiety, or an anthraquinone moiety (e.g., a carbene forming moiety).

Overall, described herein are improved methods for analyzing a biological sample comprising a nucleic acid molecule, which may have applications for in situ analysis, including immobilization of nucleic acid molecules and detection of nucleic acid molecules, such as nucleic acid concatemers.

In the following sections, additional description of various aspects of the methods, compositions, and kits disclosed herein is provided. Section II describes methods of in situ analysis of a biological sample comprising a nucleic acid molecule using probes comprising a branched linking a either: i) a detectable label, or ii) a photoactivatable functional moiety; Section III describes exemplary methods for detection and analysis of the nucleic acid molecules; and Section IV describes exemplary biological samples as well as analytes (e.g., endogenous analytes, labeling agents, or products of endogenous nucleic analytes) that can be detected using a method of analyzing a biological sample described herein (e.g., by detecting a target nucleic acid that is or is associated with the analyte). Section V provides kits according to the present disclosure, and Section VI describes exemplary applications of the present methods, compositions, and kits. As stated above, the section headings used herein are for organizational purposes only and are not to be construed as limiting the subject matter described.

### II. Methods of In Situ Analysis Using Branched Probes

This application provides, inter alia, a method for analyzing a biological sample comprising contacting the biological sample with a probe (e.g., an oligonucleotide). The probe/oligonucleotide comprises a hybridization region complementary to a target nucleic acid sequence, a detectable label or a photoactivatable functional moiety capable of covalently bonding to an endogenous molecule in the biological sample, and a linker. The linker may be a branched linker comprising a plurality of arms, that each may be coupled to one or more photoactivatable functional moieties or one or more detectable labels. An advantage of the branched linkers provided herein is the ability to accommodate a plurality of photoactivatable functional moieties and/or detectable labels, such that the probe and/or oligonucleotide itself does not need to be lengthened.

A. Branched Linkers

The probes and/or oligonucleotides provided herein may comprise a linker. In some embodiments, the linker is a branched linker. In some embodiments, the branched linker comprises a plurality of arms. In some embodiments, one or more of the arms are coupled to one or more detectable labels and/or one or more photoactivatable functional moieties. In some embodiments, the linker comprises an amidite (e.g., phosphoramidite). In some embodiments, the linker is a triple branch point linker. In some embodiments, the linker is a triple branch point amidite linker.

In some embodiments, the branched linker comprises one or more non-nucleic acid moieties. In some embodiments, the branched linker comprises one or more nucleic acid moieties. In some embodiments, the branched linker comprises one or more non-nucleic acid moieties and one or more nucleic acid moieties. In some embodiments, the one or more nucleic acid moieties comprise a homopolymeric sequence. In some embodiments, the homopolymeric sequence is a poly(T) sequence.

In some embodiments, the linker may comprise additional chemical groups to increase linearity away from the hybridization region. In some embodiments, the linker comprises an alkene.

In some embodiments, the probe and/or oligonucleotide comprises two or more branched linkers, such as any of 3, 4, 5, 6, 7, 8, 9, 10, or more, branched linkers. In some embodiments, the branched linker is coupled to the 3′ end of the hybridization region. In some embodiments, the branched linker is coupled to the 5′ end of the hybridization region. In some embodiments, the probe and/or oligonucleotide comprises a first branched linker coupled to the 3′ of the hybridization region and a second branched linker coupled to the 5′ of the hybridization region. In some embodiments, the hybridization region comprises an oligonucleotide or analog thereof. In some embodiments, the hybridization region comprises a DNA oligonucleotide. In some embodiments, the hybridization region comprises a morpholino. In some embodiments, the hybridization region comprises a DNA oligonucleotide and a morpholino.

In some embodiments, the branched linker comprises one level of branching (e.g., a first level branch moiety). In some embodiments, the branched linker comprises multiple levels of branching. In some embodiments, the branched linker comprises 1, 2, 3, 4, 5, 6, 7, 8, 9, 10, or more, levels of branching. In some embodiments, the branched linker comprises 10, 9, 8, 7, 6, 5, 4, 3, 2, or fewer, levels of branching. For example, the branched linker may comprise a first level branched moiety, a second level branched moiety, a third level branched moiety, a fourth level branched moiety, or greater.

In some embodiments, the branched linker comprises a first level branch moiety comprising n branches, wherein n is an integer. In some embodiments, n is 1, 2, 3, 4, 5, or greater. In some embodiments, n is 2, 3, or greater. In some embodiments, n is 2 or greater. In some embodiments, the first level branch moiety comprises an amidite. In some embodiments, the branched linker further comprises one or more spacers in the first level branch moiety. In some embodiments, the one or more spacers comprises an amidite.

In some embodiments, the branched linker comprises a second level branch moiety comprising m branches, wherein m is an integer. In some embodiments, m is 0, 1, 2, 3, 4, 5, or greater. In some embodiments, m is 0 or greater. In some embodiments, m is 2, 3, or greater. In some embodiments, m is 2 or greater. In some embodiments, the second level branch moiety comprises an amidite. In some embodiments, the branched linker further comprises one or more spacers in the second level branch moiety. In some embodiments, the one or more spacers comprises an amidite.

In some embodiments, the branched linker comprises a first level branch moiety comprising n branches and a second level branch moiety comprising m branches, wherein n and m are integers. In some embodiments, n is 1, 2, 3, 4, 5, or greater. In some embodiments, n is 2, 3, or greater. In some embodiments, n is 2 or greater. In some embodiments, m is 0, 1, 2, 3, 4, 5, or greater. In some embodiments, m is 0 or greater. In some embodiments, m is 2, 3, or greater. In some embodiments, m is 2 or greater. In some embodiments, n is 2 or greater, and m is 0 or greater. In some embodiments, n and m are independently 2, 3, or greater. In some embodiments, n and m are independently 2 or greater. In some embodiments, the branched linker comprises n×m branches, each coupled to one or more molecules of a detectable label or a photoactivatable functional moiety. In some embodiments, the branched linker is coupled to the 3′ end of the hybridization region. In some embodiments, the branched linker is coupled to the 5′ end of the hybridization region. In some embodiments, the branched linker is coupled to the 3′ end and the 5′ end of the hybridization region. In some embodiments wherein the 3′ or 5′ end of the hybridization region is not coupled to a branched linker, the 3′ or 5′ of the hybridization region is coupled to one or more molecules of the detectable label and/or the photoactivatable moiety.

In some embodiments, the first level branch moiety comprises an amidite. In some embodiments, the second level branch moiety comprises an amidite. In some embodiments, the first level branch moiety comprises an amidite and the second level branch moiety comprises an amidite. In some embodiments, the branched linker further comprises one or more spacers in the first level branch moiety. In some embodiments, the branched linker further comprises one or more spacers in the second level branch moiety. In some embodiments, the branched linker further comprises one or more spacers in the first level branch moiety and one or more spacers in the second level branch moiety. In some embodiments, the branched linker further comprises one or more spacers between the first level branch moiety and the second level branch moiety. In some embodiments, the one or more spacers comprises an amidite.

In some embodiments, the branched linker can comprise or be generated using a flexible spacer molecule and/or a branch point molecule. In some embodiments, the branched linker can comprise or be generated using a linear spacer amidite, such as 12-(4,4′-Dimethoxytrityloxy)dodecyl-1-[(2-cyanoethyl)-(N,N-diisopropyl)]-phosphoramidite. In some embodiments, the branched linker can comprise or be generated using a double branch point amidite, such as 1,3-bis-[5-(4,4′-dimethoxytrityloxy)pentylamido]propyl-2-[(2-cyanoethyl)-(N,N-diisopropyl)]-phosphoramidite. In some embodiments, the branched linker can comprise or be generated using a triple branch point amidite, such as Tris-2,2,2-[3-(4,4′-dimethoxytrityloxy)propyloxymethyl]ethyl-[(2-cyanoethyl)-(N,N-diisopropyl)]-phosphoramidite.

In some embodiments, the branched linker can comprise or be generated using any one or more of more of:

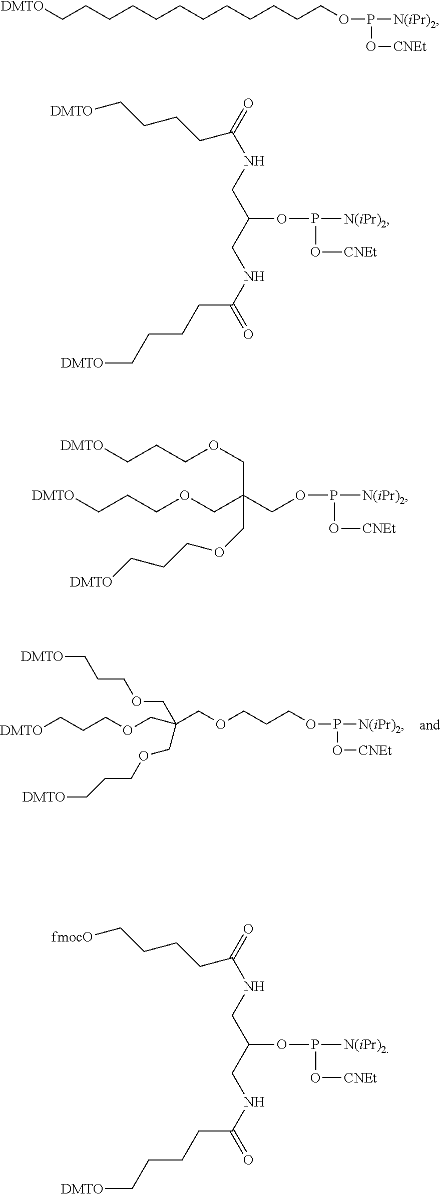

In some embodiments, the branched linker can comprise a plurality of arms and is not a dendrimer. In some embodiments, the branched linker has more polarity from an initiation point than a dendrimer which comprises arms and branch points that are substantially equal in all directions from a central initiation point.

In some embodiments, the branched linker of the probe or oligonucleotide may be covalently coupled to the hybridization region complementary to the target nucleic acid sequence. In some embodiments, the branched linker is covalently bonded to the 5′-end of the hybridization region complementary to the target nucleic acid sequence. In some embodiments, the branched moiety is generated via a reaction between 5′-nucleoside of the hybridization region complementary to the target nucleic acid sequence and a branching reagent. In some such embodiments, the branching reagent is a phosphoramidite molecule comprising two or more arms. In some embodiments, the branching agent includes two arms. In some embodiments, the branching agent includes three arms. In some embodiments, the branching agent is a long trebler phosphoramidite. In some embodiments, the hybridization region complementary to the target nucleic acid sequence comprises a DNA oligonucleotide and/or a morpholino.

In some embodiments, each of the arms of the branching agent (e.g., long trebler phosphoramidite) of the probe is protected with a protecting group. In some embodiments, the protecting group is a 4,4′-dimethoxytrityl (DMT) group. In some embodiments, the protecting group is a fluorenylmethoxycarbonyl (Fmoc) group. In some embodiments, the protecting group (e.g., DMT) is removed prior to reaction with a spacer, as set forth herein. In some embodiments, the protecting group (e.g., DMT group or Fmoc group) is removed with a weak or mild acid or base prior to reaction with the spacer, detectable label or agent comprising a photoactivatable functional moiety. In some embodiments, the protecting group (e.g., DMT group) is removed with dichloroacetic acid. In some embodiments, the protecting group (e.g., DMT group) is removed with trichloroacetic acid. In some embodiments, the protecting group (e.g., FMOC group) is removed with a base such as piperidine.

In embodiments with branched phosphoramidite branched linkers, such as those depicted in the preceding paragraph, the N(iPr)₂ group of the phosphoramidite may be cleaved following nucleophilic attack of the nucleoside at the 5′-end of the hybridization region of the probe or oligonucleotide. In some embodiments, the reaction between the hybridization region and the phosphoramidite is catalyzed by tetrazole.

In one embodiment, following reaction between the nucleoside at the 5′-end of the hybridization region and

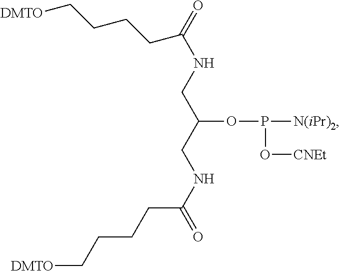

the resultant branched linker (prior to removal of the DMT protecting groups and the —OCNEt group) has the structure depicted below:

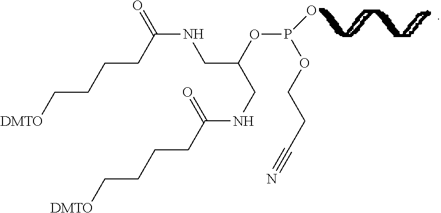

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate a compound having the structure depicted below:

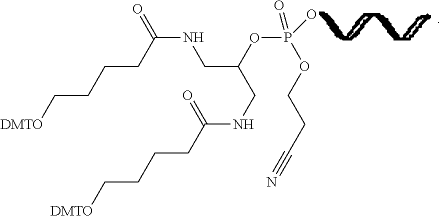

In some embodiments, oxidation is conducted with iodine (I₂), which increases the valency of the phosphorous from 3 to 5. Following removal of the DMT groups, the molecule depicted above can be added directly to a spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In some embodiments, the branched linker (prior to removal of the DMT protecting groups and the —OCNEt group) has the structure depicted below:

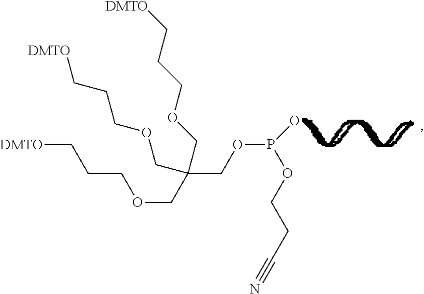

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate a compound has the structure depicted below:

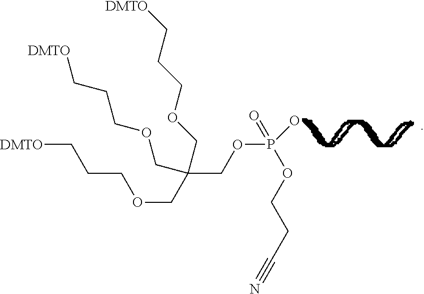

In some embodiments, oxidation is conducted with iodine (I₂), which increases the valency of the phosphorous from 3 to 5. Following removal of the DMT groups, the molecule depicted above can be added directly to a spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In some embodiments, the branched linker (prior to removal of the DMT protecting groups and the —OCNEt group) has the structure depicted below:

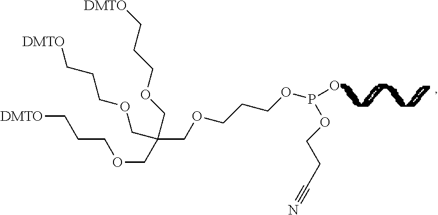

wherein [symbol] represents the hybridization region complementary to the target nucleic acid. Following removal of the DMT groups, the molecule depicted above can be added directly to a spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In some embodiments, the resultant branched linker (prior to removal of the DMT protecting groups and the —OCNEt group) can be oxidized to generate the compound depicted below:

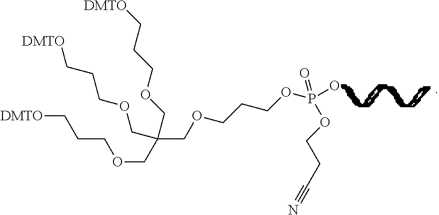

In some embodiments, oxidation is conducted with iodine (I₂), which increases the valency of the phosphorous from 3 to 5. Following removal of the DMT groups, the molecule depicted above can be added directly to spacer, detectable label or agent comprising a photoactivatable functional moiety, thereby generating the probe or oligonucleotide. In some embodiments, the —OCNEt group is cleaved prior to or following covalent attachment of the spacer, detectable label or agent comprising the photoactivatable functional moiety.

In any of the embodiments herein, the branched linker of the probe can comprise a flexible spacer molecule. In some embodiments, the flexible spacer is between one or more arms of the branched linker and the detectable label or the agent comprising a photoactivatable functional moiety. In some embodiments, the spacer provides a long hydrophilic attachment between one or more arms of the branched linker and the detectable label or agent comprising the photoactivatable functional moiety. In some embodiments, the spacer is a tetraethelyene glycol (TEG) spacer. In some embodiments, the spacer is a hexaetheylene glycol (HEG) spacer. In some embodiments, the spacer is a C6, C12, or C18 spacer. In some embodiments, the spacer is an 18-atom-hexa-ethtleneglycol spacer. In some embodiments, the spacers are added to one or more arms of the branched linkers following removal of the protecting groups (e.g., DMT groups) of the branched linkers. In some embodiments, the spacers can be covalently attached to the detectable labels or agent comprising the photoactivatable functional moiety.

In one embodiment, the probe or oligonucleotide has the structure depicted below:

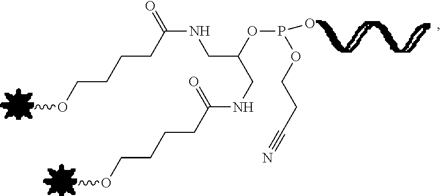

wherein [symbol] represents the hybridization region complementary to the target nucleic acid, PGP-111 [symbol] represents the spacer and [symbol] represents the detectable labels or agent comprising the photoactivatable functional moiety.

In one embodiment, the probe or oligonucleotide has the structure depicted below:

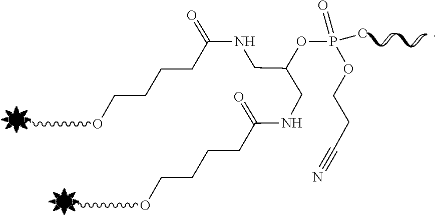

In one embodiment, the probe or oligonucleotide has the structure depicted below:

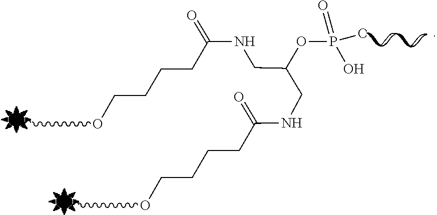

In one embodiment, the probe or oligonucleotide has the structure depicted below:

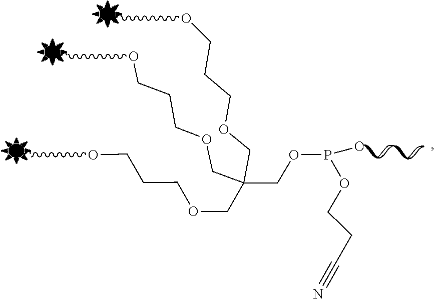

wherein [symbol] represents the hybridization region complementary to the target nucleic acid, [symbol] represents the spacer and [symbol] represents the detectable labels or agent comprising the photoactivatable functional moiety.

In one embodiment, the probe or oligonucleotide has the structure depicted below:

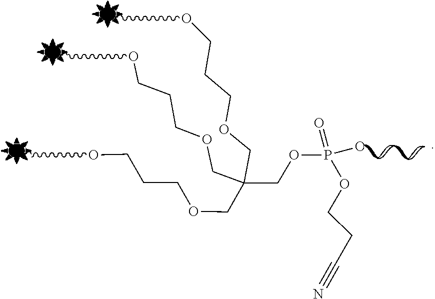

In one embodiment, the probe or oligonucleotide has the structure depicted below:

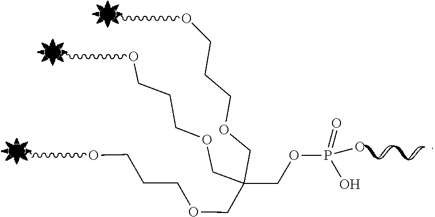

In one embodiment, the probe or oligonucleotide has the structure depicted below:

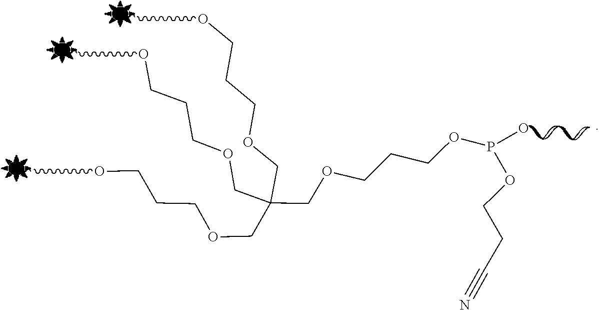

wherein [symbol] represents the hybridization region complementary to the target nucleic acid, [symbol] represents the spacer and [symbol] represents the detectable labels or agent comprising the photoactivatable functional moiety.

In one embodiment, the probe or oligonucleotide has the structure depicted below:

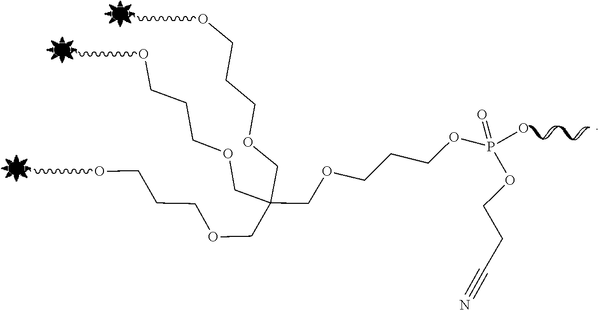

In one embodiment, the probe or oligonucleotide has the structure depicted below:

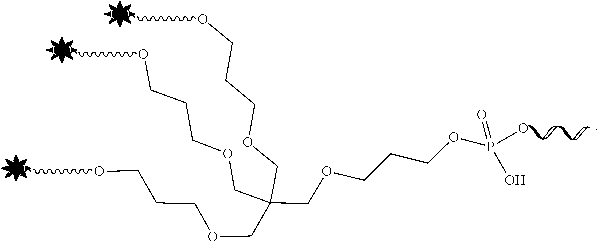

In some embodiments, the probe does not comprise a spacer between the detectable label or agent comprising the photoactivatable functional moiety and the arms of the branched linker. In such embodiments, the detectable label or agent comprising the photoactivatable functional moiety may be covalently attached directly to the arms of the branched linkers.

B. Methods Involving Detectable Labels

In some aspects, provided herein are methods and compositions for analyzing a biological sample (e.g., cell or a tissue sample) comprising contacting a biological sample with a probe comprising a detectable label. In some embodiments, the method comprises contacting a biological sample with a probe comprising: i) a hybridization region complementary to a target nucleic acid sequence, ii) a plurality of detectable labels, and iii) a branched linker coupled to the hybridization region. In some embodiments, the branched linker comprises a plurality of arms. In some embodiments, one or more of the arms are coupled to one or more detectable labels, thereby hybridizing the probe to a target molecule at a location in the biological sample. In some embodiments, the target molecule comprises one or more copies of the target nucleic acid sequence. For example, the target molecule may be a RCP. In some aspects, the method further comprises performing an amplification reaction to form the target molecule (e.g., RCP). Finally, the method comprises detecting signals associated with the plurality of detectable labels at the location in the biological sample.

In any of the embodiments herein, the hybridization region of the probe that is complementary to the target nucleic acid sequence may be prepared synthetically. In some embodiments, the hybridization region complementary to the target nucleic acid sequence is prepared synthetically by coupling a nucleoside phosphoramidite to a growing oligomer strand in the 3′-5′ direction. In some embodiments, the DNA synthesis is carried out on a solid support (e.g., universal support).

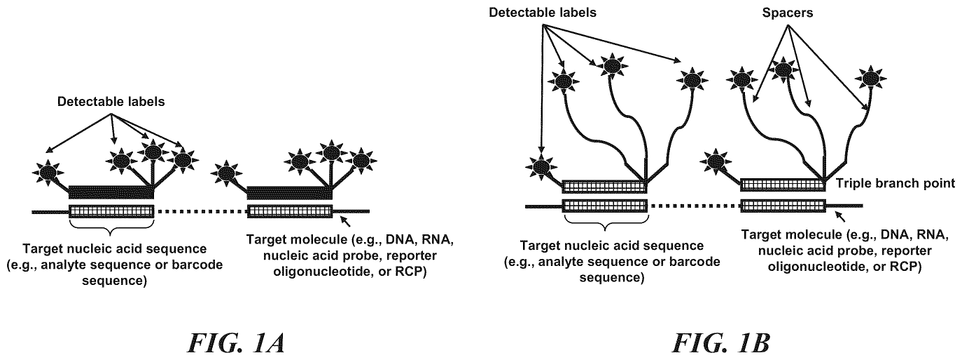

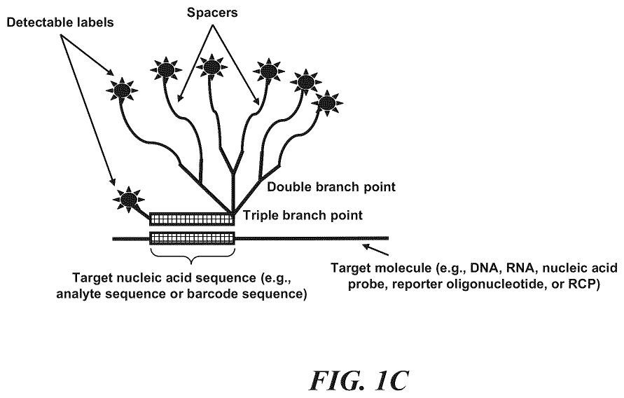

FIGS. 1A-1C show exemplary probes each comprising a branched linker with a plurality of arms, each coupled to a detectable label (e.g., fluorescent moieties). The exemplary probe comprises a hybridization region complementary to a target nucleic acid sequence in a DNA concatemer. The target nucleic acid sequence may be in a target molecule that can be a RCP. In some embodiments, the target nucleic acid sequence may be in a target molecule that is a nucleic acid probe (e.g., an intermediate probe) that directly or indirectly binds to a DNA concatemer. In some cases, the intermediate probe comprises a 3′ and/or a 5′ overhang and the target nucleic acid sequence may be in the 3′ overhang and/or the 5′ overhang. This method allows for enhanced detection of nucleic acid targets in situ by producing a stronger detection signal without increasing the length of the detection probe.

(i) Target Nucleic Acid Sequence and Target Molecules

The target sequences that are complementary to the hybridization region of the probes described herein are comprised in a target molecule that comprises one or more copies of the target sequence. In some embodiments, the target molecule is a DNA concatemer (e.g., nucleic acid concatemer). In some embodiments, the target molecule is an intermediate probe that directly or indirectly binds to a DNA concatemer.

In some embodiments, the target molecule is a product (e.g., an amplification product, such as a RCP) of the target sequence (e.g., analyte) in the biological sample. In some embodiments, the DNA concatemer is a rolling circle amplification (RCA) product (RCP). RCA may comprise contacting the biological sample with one or more probes (e.g., a circular probe or circularizable probe or probe set) to produce a RCP. In some embodiments, the RCP is generated in situ in the biological sample. In some embodiments, the one or more probes or probe sets comprise a circular probe, or a circularizable probe or probe set. In some embodiments, the circular probe or the circularizable probe or probe set is or comprises an oligonucleotide. In some embodiments, the RCP is a product of a nucleic acid molecule in the biological sample. In some embodiments, the RCP is a product of a circular or circularizable probe or probe set that hybridizes to the nucleic acid molecule. In some embodiments, the nucleic acid molecule is genomic DNA, mRNA, or cDNA. In some embodiments, the RCP is a product of a reporter oligonucleotide of a labelling agent that directly or indirectly binds to an analyte in the biological sample. In some embodiments, the RCP is a product of a circular or circularizable probe or probe set that hybridizes to the reporter oligonucleotide. In some embodiments, the labelling agent further comprises a binding moiety that directly or indirectly binds to a nucleic acid analyte and/or a non-nucleic acid analyte in the biological sample. RCA, and RCPs thereof, is further described in Section. III.B.iii.

In some embodiments, a RCP is generated from a nucleic acid analyte. For example, the nucleic acid analyte may be DNA, ssDNA, or RNA. In some embodiments, the analyte is a non-nucleic acid analyte (e.g., a protein). In some embodiments, the nucleic acid concatemer is generated from a labeling agent that directly or indirectly binds to an analyte in the biological sample. Examples of labeling agents are described in Section III.B.ii.

In some embodiments, the target sequence is a nucleic acid analyte or a non-nucleic acid analyte, and the labeling agent comprises a reporter oligonucleotide and an analyte-binding moiety coupled thereto. In some embodiments, the DNA concatemer (e.g., RCP) comprises nucleic acids. In some embodiments, the RCP contains natural and unnatural nucleotides. For example, the RCP may comprise modified nucleotides, non-nucleotides, or synthetic nucleotides. Nucleotides amendable to the present application include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Additional suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. In some embodiments, the modified nucleotides are amine-modified nucleotides.

The RCP may comprise tandem repeats. In some embodiments, “tandem repeats” refers to a sequence of two or more nucleic acid residues that is repeated in an adjacent manner on the RCP. For example, the tandem repeats may be incorporated into the RCP via a primer used for amplification (e.g., RCA). In some embodiments, the tandem repeats in the RCP do not comprise a functional group capable of crosslinking with a second functional group. In some embodiments, the tandem repeats in the RCP are free of nucleic acid residues comprising functional group B. In some embodiments, the tandem repeats in the RCP are free of modified nucleic acid residues. In some embodiments, the modified nucleic acid residues are amine-modified. In some embodiments, the tandem repeats in the RCP are composed entirely of naturally occurring nucleic acid residues.

In some embodiments, the RCP is between about 1 and about 85 kilobases in length, such as between any of about 1 and about 15 kilobases, about 10 and about 30 kilobases, about 20 and about 40 kilobases, about 30 and about 50 kilobases, about 40 and about 60 kilobases, about 50 and about 70 kilobases, and about 60 and about 85 kilobases in length. In some embodiments, the nucleic acid concatemer is at least 1 kilobase in length, such as any of about 15, 25, 35, 45, 55, 65, or 85 kilobases in length. In some embodiments, the RCP is more than 85 kilobases in length.

In some embodiments, the RCP comprises a barcode sequence corresponding to an analyte in the biological sample, and the RCP is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length.

In some embodiments, the RCP forms a nanoball such as one having a diameter between about 0.1 μm and about 3 μm. The nanoball may be a product of amplification, such as RCA. In some embodiments, the nanoball has a diameter of between about 0.1 μm and about 4 μm, such as between any of about 0.1 μm and about 0.5 μm, about 0.2 μm and about 2 μm, about 1 μm and about 3 μm, and about 2 μm and about 4 μm. In some embodiments, the nanoball diameter is at least about 0.1 μm, such as at least any of about 0.2 μm, 0.5 μm, 1 μm, 1.5 μm, 2 μm, 2.5 μm, 3 μm, 3.5 μm, and 4 μm. In some embodiments, the nanoball diameter is less than about 500 nm. In some embodiments, the nanoball diameter is less than about 250 nm.

In some embodiments, the RCP comprises more than one copy of a unit sequence corresponding to the RCA template. In some embodiments, the RCP comprises between about 10 and about 100, between about 100 and about 1,000, between about 1,000 and about 5,000, between about 5,000 and about 10,000, or more than 10,000 copies of a unit sequence corresponding to the rolling circle amplification template.

In some embodiments, the biological sample comprises a plurality of RCPs. In some embodiments, about 90% (e.g., about 80%, 85%, 90%, 91%, 92%, 93%, 94%, 95%, 96%, 97%, 98%, 99%) of the plurality of RCPs in the biological sample have a diameter of less than about 500 nm. In some embodiments, about 90% (e.g., about 80%, 85%, 90%, 91%, 92%, 93%, 94%, 95%, 96%, 97%, 98%, 99%) of the plurality of RCPs in the biological sample have a diameter of less than about 250 nm.

(a) Amplification with Primer Comprising a Photoactivatable Functional Moiety

As described herein, there is provided a method for analyzing a biological sample, comprising: a) contacting the biological sample with a circular probe or circularizable probe or probe set that hybridizes to a target analyte in the biological sample; b) generating a rolling circle amplification product (RCP) in situ in the biological sample using the circular probe or a circularized probe generated from the circularizable probe or probe set as a template; c) contacting the biological sample with a probe comprising: i) a hybridization region complementary to a target nucleic acid sequence in the RCP or in an intermediate probe that hybridizes to the RCP, ii) a plurality of detectable labels, and iii) a branched linker coupled to the hybridization region, wherein the branched linker comprises a plurality of arms each coupled to one or more detectable labels, thereby hybridizing the probe to the RCP or the intermediate probe in the biological sample; and d) detecting signals associated with the plurality of detectable labels to detect the target analyte in the biological sample.

In some embodiments, the RCA primed using a primer comprising a photoactivatable functional moiety capable of covalently bonding to an endogenous molecule in the biological sample, thereby immobilizing the primer and a RCP thereof in the biological sample. In some embodiments, the endogenous molecule is a protein or a nucleic acid.

In some embodiments, the photoactivatable functional moiety is a diazirine moiety, an aryl azide moiety, a benzophenone moiety, or an anthraquinone moiety. In some embodiments, the photoactivatable functional moiety is a carbene forming moiety.

In some embodiments, the primer comprises a primer region that hybridizes to the circular or circularized probe and a linker coupling the functional moiety to the primer region. In some embodiments, the linker is a branched linker, such as any of the branched linkers described herein, comprising a plurality of arms each coupled to one or more of the photoactivatable functional moieties. In some embodiments, the linker may comprise additional chemical groups to increase linearity away from the primer region. In some embodiments, the linker comprises an alkene. In some embodiments, the primer comprises at least about 3, at least about 5, at least about 10, at least about 15, at least about 20, or more than 20 of the photoactivatable functional moiety.

In some embodiments, the method comprises reacting the photoactivatable functional moiety with the endogenous molecule prior to, during, or after generating the RCP in the biological sample. In some embodiments, the method comprises reacting the photoactivatable functional moiety with the endogenous molecule prior to generating the RCP in the biological sample. In some embodiments, the method comprises reacting the photoactivatable functional moiety with the endogenous molecule during the generating the RCP in the biological sample. In some embodiments, the method comprises reacting the photoactivatable functional moiety with the endogenous molecule after generating the RCP in the biological sample. In some embodiments, reacting the photoactivatable functional moiety with the endogenous molecule immobilizes the RCP in the biological sample via the primer.

In some embodiments, the photoactivatable functional moiety is activated by exposing the biological sample to UV light. In some embodiments, the biological sample is exposed to a wavelength of light between about 100 nm and about 450 nm, such as between about 350 nm and about 450 nm. In some embodiments, upon exposure to UV light, the activated photoactivatable functional moiety reacts with the endogenous molecule in the biological sample. In some embodiments, the endogenous molecule is a polypeptide and the activated photoactivatable functional moiety reacts with an amino acid side chain or a peptide backbone of the polypeptide.

(ii) Detectable Labels

The methods and compositions provided herein may comprise a probe comprising a plurality of detectable labels. In some embodiments, the detectable label comprises a fluorescent moiety. In some embodiments, each of the plurality of detectable labels comprises a fluorescent moiety. Additional detectable moieties and further description of fluorescent moieties, as well as detection thereof, are described in Section IV.

In some embodiments, the probe comprises a plurality of the same fluorescent moieties. For example, in some embodiments, each of the plurality of detectable labels comprises the same fluorescent moiety. In some embodiments, the plurality of detectable labels comprise different fluorescent moieties. In some embodiments, each of the plurality of detectable labels comprises different fluorescent moieties. In some embodiments, the probe comprises at least about 3, at least about 5, at least about 10, at least about 15, at least about 20, or more than 20 molecules of the same fluorescent moiety. In some embodiments, the probe comprises at least about 3, at least about 5, at least about 10, at least about 15, at least about 20, or more than 20 molecules of different fluorescent moieties.

C. Methods Involving Photoactivatable Functional Moieties

In some aspects, provided herein is a method for analyzing a biological sample, comprising: a) contacting the biological sample with a circular probe or circularizable probe or probe set that hybridizes to a target analyte in the biological sample; b) generating a rolling circle amplification (RCA) product (RCP) in situ in the biological sample using the circular probe or a circularized probe generated from the circularizable probe or probe set as a template; c) contacting the biological sample with an oligonucleotide comprising: i) a hybridization region complementary to a target nucleic acid sequence in the RCP or in an intermediate probe that directly or indirectly binds to the RCP, ii) one or more photoactivatable functional moieties, and iii) a linker coupled to the hybridization region, wherein the linker comprises one or more arms, and wherein the one or more arms are coupled to one or more photoactivatable functional moieties, thereby hybridizing the oligonucleotide to the RCP or the intermediate probe in the biological sample; d) activating the one or more functional moieties by exposing the biological sample to UV light, wherein following exposure to the UV light, the one or more functional moieties react with one or more endogenous molecules in the biological sample, thereby immobilizing the RCP in the biological sample; and e) detecting the target analyte in the biological sample. In some embodiments, detecting the target analyte comprises contacting the biological sample with a detectable probe that directly or indirectly binds to the oligonucleotide or a product thereof.

In some aspects, provided herein are methods and compositions for analyzing a biological sample (e.g., cell or a tissue sample) comprising contacting a biological sample with a probe comprising a photoactivable function moiety. In some embodiments, the method comprises contacting a biological sample with an oligonucleotide comprising: i) a hybridization region complementary to a target nucleic acid sequence, ii) a photoactivatable functional moiety capable of covalently bonding to an endogenous molecule in the biological sample, and iii) a linker coupling the functional moiety to the hybridization region, thereby hybridizing the oligonucleotide to the target molecule at a location in the biological sample. The photoactivatable functional moiety may be reacted with the endogenous molecule in the biological sample, thereby immobilizing the oligonucleotide in the biological sample. Then, the biological sample may be contacted with a detectable probe that directly or indirectly binds to the oligonucleotide or a product thereof. Finally, a signal associated with the detectable probe at the location in the biological sample can be detected.

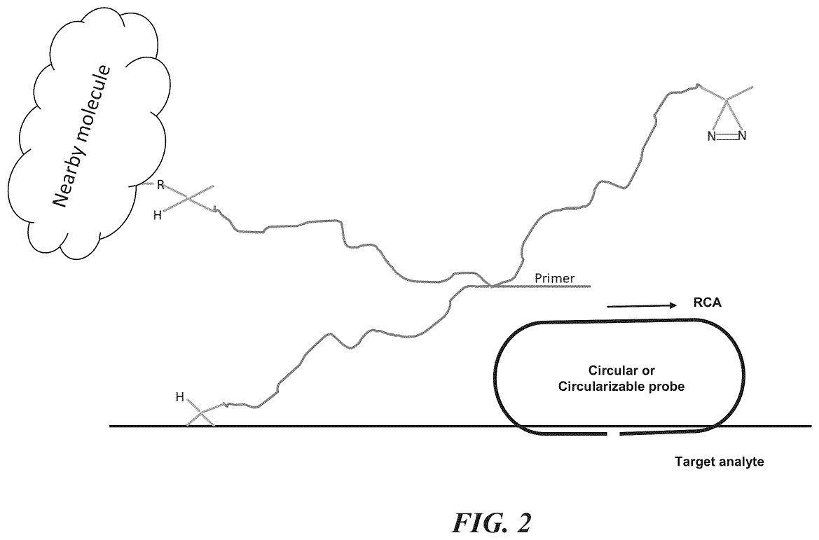

FIG. 2 shows an exemplary oligonucleotide comprising a branched linker with a plurality of arms, each coupled to a photoactivatable functional moiety (e.g., a diazirine moieties) capable of coupling to an endogenous molecule in a biological sample. The exemplary oligonucleotide may be an amplification primer, and comprises a hybridization region complementary to a target nucleic acid sequence in a target molecule. As illustrated in FIG. 2, the target molecule can be a nucleic acid probe that hybridizes to a genomic DNA, mRNA, or cDNA, or a product of the nucleic acid probe, such as a circular or circularizable probe or probe set, the oligonucleotide primes rolling circle amplification of the circular probe or a circularized probe generated from the circularizable probe or probe set, and the product of the oligonucleotide is a RCP. In some embodiments, the oligonucleotide is coupled to the photoactivatable function moiety at the 5′ end and has an available 3′ end for extension in the amplification reaction. The endogenous molecule may be a protein or a nucleic acid. In FIG. 2, the R group represents an amino acid side chain of a protein that reacts with a carbene intermediate generated by photoactivation of the diazirine moiety. In the figure, only one of the diazirine moieties are shown to be reactive. It will be understood that all of the diazirine moieties can be photoactivated to form carbene intermediates that react with an endogenous molecule in a biological sample. This method allows for the immobilization of the oligonucleotide in the biological sample, thereby preventing diffusion of the product of the oligonucleotide. In some aspects, this method provides for improved resolution of nucleic acid targets in situ.

In some embodiments, the oligonucleotide is prepared by reacting functional moieties on one or more of the arms of the linker bound to the hybridization domain with an agent comprising a photoactivatable functional moiety such as a diazirine moiety, an aryl azide moiety, a benzophenone moiety, or an anthraquinone moiety. In some embodiments, the linker covalently bonded to photoactivatable functional is a linker with a single arm (i.e., not branched). In some embodiments, the hybridization probe is generated via a reaction between 5′-nucleoside of the hybridization region complementary to the target nucleic acid sequence and a single-armed linker. In some embodiments, the single-armed linker is a phosphoramidite compound. As set forth with respect to the branched phosphoramidite linkers, following reaction between the 5′-nucleoside of the hybridization region and the single-armed phosphoramidite linker, the protecting group (e.g., DMT group) of the phosphoramidite linker can be chemically removed and the resultant unprotected phosphoramidite can be reacted directly with an agent comprising the photoactivatable functional moiety or with a spacer. In some embodiments, following covalent coupling of the phosphoramidite and the 5′-nucleoside of the hybridization region, oxidation is conducted with iodine (I₂), which increases the valency of the phosphorous on the phosphoramidite from 3 to 5.

In one embodiment, the single-armed linker used in the coupling reaction is 9-O-Dimethoxytrityl-triethylene glycol, 1-[(2-cyanoethyl)-(N,N-diisopropyl)]-phosphoramidite, also referred to as spacer phosphoramidite 9, which has the structure depicted below.

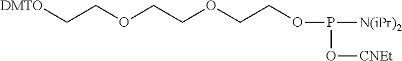

In another embodiment, the single-armed linker used in the coupling reaction is 18-O-Dimethoxytritylhexaethyleneglycol, 1-[(2-cyanoethyl)-(N,N-diisopropyl)]-phosphoramidite, also referred to as spacer phosphoramidite 9, which has the structure depicted below:

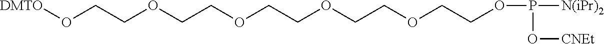

In another embodiment, the single-armed linker used in the coupling reaction is 12-(4,4′-Dimethoxytrityloxy)dodecyl-1-[(2-cyanoethyl)-(N,N-diisopropyl)]-phosphoramidite, also referred to as spacer C12 CE phosphoramidite, which has the structure depicted below:

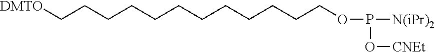

In another embodiment, the single-armed linker used in the coupling reaction is 3-(4,4′-Dimethoxytrityloxy)propyl-1-[(2-cyanoethyl)-(N,N-diisopropyl)]-phosphoramidite, also referred to as spacer phosphoramidite C3, which has the structure depicted below:

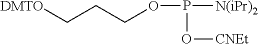

In some embodiments, the linker used in the coupling reaction is a branched linker with multiple arms, as described herein. In some such embodiments, the branched linker with multiple arms is a branched phosphoramidite linker.

In one embodiment where the oligonucleotide comprises a branched linker, all of the protecting groups (e.g., DMT or FMOC) are removed from the arms of branched linker prior to reaction with the agent comprising the photoactivatable functional moiety. The deprotected arms of the linker can then be reacted with the agent comprising a photoactivatable functional moiety, hence generating the photoactivatable probe. Alternatively, as set forth herein, one or more spacers can be added to the terminal ends of the arms of branched linkers following deprotection of the branched linkers. In such embodiments, the spacers may contain a functional moiety capable of reacting with the agent comprising the photoactivatable functional moiety. Reaction between the functional moiety on the spacer with the agent comprising a photoactivatable functional moiety generates the probe.

In some embodiments where the oligonucleotide comprises a branched linker, not all of the arms of the branched linker will include the photoactivatable functional moiety. For instance, in some embodiments where the branched linker includes two or three branches, only one of the arms will include the photoactivatable functional moiety. In these embodiments, the probes can be generated by selectively removing one of the protecting groups of the branched linker prior to reaction with the agent comprising the photoactivatable functional moiety. For instance, in a branched linker that includes two arms, where one arm is protected by DMT and the other arm is protected by FMOC, the FMOC can first be selectively removed and the DMT can be left intact. The resultant compound can then be added directly to the agent comprising the photoactivatable functional moiety or to a spacer. Following attachment of the agent comprising the photoactivatable functional moiety to the first arm, the DMT group of the second branch can be removed, hence generating the photoactivatable probe.

In some embodiments, the agent comprising the photoactivatable functional moiety is a diazirine moiety. In some such embodiments, the agent comprising the photoactivatable functional moiety is succinimidyl 4,4′-axipentanoate (NHS-Diazirine), sulfo-NHS-Diazirine, sulfosuccinimidyl 4,4′-azipentanoate (Sulfo-NHS-Diazirine), succinimidyl 6-(4,4′-azipentanamido)hexanoate (NHS-LC-Diazirine), succinimidyl 2-((4,4′-azipentanamido)ethyl)-1,3′-dithiopropionate (NHS-SS-Diazirine), or sulfo-NHS-SS-diazirine.

(i) Target Nucleic Acid Sequence, Target Molecules, and Oligonucleotides

The target sequences that are complementary to the hybridization region of the probes described herein are comprised in a target molecule that comprises the target sequence. In some embodiments, the target molecule is a genomic DNA, mRNA, or cDNA, or a product thereof. In some embodiments, the target molecule is a nucleic acid probe that hybridizes to a genomic DNA, mRNA, or cDNA, or a product of the nucleic acid probe. In some embodiments, the target molecule is a circular or circularizable probe or probe set, the oligonucleotide primes RCA of the circular probe or a circularized probe generated from the circularizable probe or probe set, and the product of the oligonucleotide is a RCP. In some embodiments, the endogenous molecule is a protein or a nucleic acid.

In some embodiments, the oligonucleotide is a probe that comprises one or more DNA bases. In some embodiments, the oligonucleotide is a probe that comprises one or more RNA bases. In some embodiments, the oligonucleotide is a probe that comprises one or more DNA bases and one or more RNA bases. In some embodiments, the probe is a circular probe. In some embodiments, the circular probe is a circularized probe generated from a circularizable probe or probe set hybridized to a nucleic acid (e.g., a genomic DNA, an mRNA, a cDNA, or a probe) in the biological sample. In some embodiments, the probe may be used to amplify an analyte, such as a nucleic acid molecule analyte (e.g., any of the analytes described in Section IIIB, below), in the biological sample to generate nucleic acid concatemers, such as isothermal enzymatic amplification, inducing but not limited to RCA. RCA is further described in Section III.B.iii, below.

In some embodiments, the oligonucleotide is a circular probe or circularizable probe or probe set. For instance, a circularizable probe or probe set disclosed herein can comprise a padlock probe that does require gap filling to circularize upon hybridization to a template (e.g., a target nucleic acid and/or a probe such as a splint), a gapped padlock probe (e.g., one that require gap filling to circularize upon hybridization to a template), an L-shaped probe (e.g., one that comprises a target recognition sequence and a 5′ or 3′ overhang upon hybridization to a target nucleic acid or a probe), a U-shaped probe (e.g., one that comprises a target recognition sequence, a 5′ overhang, and a 3′ overhang upon hybridization to a target nucleic acid or a probe), a V-shaped probe (e.g., one that comprises at least two target recognition sequences and a linker or spacer between the target recognition sequences upon hybridization to a target nucleic acid or a probe), a probe or probe set for proximity ligation (such as those described in U.S. Pat. Nos. 7,914,987 and 8,580,504 incorporated herein by reference in their entireties, and probes for Proximity Ligation Assay (PLA) for the simultaneous detection and quantification of nucleic acid molecules and protein-protein interactions), or any suitable combination thereof. In some embodiments, a circularizable probe or probe set disclosed herein can comprise a probe that is ligated to itself or another probe using DNA-templated and/or RNA-templated ligation. In some embodiments, a circularizable probe or probe set disclosed herein can be a DNA molecule and can comprise one or more other types of nucleotides, modified nucleotides, and/or nucleotide analogues, such as one or more ribonucleotides. In some embodiments, the ligation can be a DNA ligation on a DNA template. In some embodiments, the ligation can be a DNA ligation on an RNA template, and the probes can comprise RNA-templated ligation probes. In some embodiments, a circularizable probe or probe set disclosed herein can comprise a padlock-like probe or probe set. In some embodiments, a nucleic acid probe disclosed herein is part of a SNAIL (Splint Nucleotide Assisted Intramolecular Ligation) probe set, such as one described in US 2019/0055594 or US 2021/0164039 which are incorporated herein by reference in their entireties. In some embodiments, a nucleic acid probe disclosed herein is part of a PLAYR (Proximity Ligation Assay for RNA) probe set, such as one described in US 2016/0108458 which is incorporated herein by reference in its entirety. In some embodiments, a nucleic acid probe disclosed herein is part of a PLISH (Proximity Ligation in situ Hybridization) probe set, such as one described in US 2020/0224243 which is incorporated herein by reference in its entirety. Any suitable combination of the probe designs described herein can be used.

Any suitable circularizable probe or probe set, or indeed more generally circularizable reporter molecules, may be used to generate the RCA template which is used to generate the RCA product. For example, a circularizable probe or probe set may be used to generate a circular nucleic acid comprising a target hybridization region. The target hybridization region in the RCA product can comprise an identifying sequence, such as a sequence of a nucleic acid analyte or probe to which the circularizable probe or probe set hybridizes, or a barcode sequence. The circularizable probe or reporter (the RCA template) can be in the form of a linear molecule having ligatable ends which may circularized by ligating the ends together directly or indirectly, e.g. to each other, or to the respective ends of an intervening (“gap”) oligonucleotide or to an extended 3′ end of the circularizable RCA template. A circularizable template may also be provided in two or more parts, namely two or more molecules (e.g. oligonucleotides) which may be ligated together to form a circle. When said RCA template is circularizable it is circularized by ligation prior to RCA. Ligation may be templated using a ligation template, and in the case of padlock and molecular inversion probes and such like the target analyte may provide the ligation template, or it may be separately provided. The circularizable RCA template (or template part or portion) will comprise at its respective 3′ and 5′ ends regions of complementarity to corresponding cognate complementary regions (or binding sites) in the ligation template, which may be adjacent where the ends are directly ligated to each other, or non-adjacent, with an intervening “gap” sequence, where indirect ligation is to take place.

In the case of padlock probes, in one embodiment the ends of the padlock probe may be brought into proximity to each other by hybridization to adjacent sequences on a target nucleic acid molecule (such as a target analyte), which acts as a ligation template, thus allowing the ends to be ligated together to form a circular nucleic acid molecule, allowing the circularized padlock probe to act as a template for an RCA reaction. In such an example, the terminal sequences of the padlock probe which hybridize to the target nucleic acid molecule will be specific to the target analyte in question, and will be replicated repeatedly in the RCA product. They may therefore act as a marker sequence indicative of that target analyte. Accordingly, it can be seen that the marker sequence in the RCA product may be equivalent to a sequence present in the target analyte itself. Alternatively, a marker sequence (e.g. tag or barcode sequence) may be provided in the non-target complementary parts of the padlock probe. In still a further embodiment, the marker sequence may be present in the gap oligonucleotide which is hybridized between the respective hybridized ends of the padlock probe, where they are hybridized to non-adjacent sequences in the target molecule. Such gap-filling padlock probes are akin to molecular inversion probes.

In some embodiments, similar circular RCA template molecules can be generated using molecular inversion probes. Like padlock probes, these are also typically linear nucleic acid molecules capable of hybridizing to a target nucleic acid molecule (such as a target analyte) and being circularized. The two ends of the molecular inversion probe may hybridize to the target nucleic acid molecule at sites which are proximate but not directly adjacent to each other, resulting in a gap between the two ends. The size of this gap may range from only a single nucleotide in some embodiments, to larger gaps of 100 to 500 nucleotides, or longer, in other embodiments. Accordingly, it is necessary to supply a polymerase and a source of nucleotides, or an additional gap-filling oligonucleotide, in order to fill the gap between the two ends of the molecular inversion probe, such that it can be circularized.

As with the padlock probe, the terminal sequences of the molecular inversion probe which hybridize to the target nucleic acid molecule, and the sequence between them, will be specific to the target analyte in question, and will be replicated repeatedly in the RCA product. They may therefore act as a marker sequence indicative of that target analyte. Alternatively, a marker sequence (e.g. tag or barcode sequence) may be provided in the non-target complementary parts of the molecular inversion probe.

In some embodiments, the probes disclosed herein may be invader probes, e.g., for generating a circular nucleic acid such as a circularized probe. In some aspects, such may be useful for detection of single nucleotide polymorphisms. The detection method of the present disclosure may, therefore, be used in the detection of a single nucleotide polymorphism, or indeed any variant base, in the target nucleic acid sequence. Probes for use in such a method may be designed such that the 3′ ligatable end of the probe is complementary to and capable of hybridizing to the nucleotide in the target molecule which is of interest (the variant nucleotide), and the nucleotide at the 3′ end of the 5′ additional sequence at the 5′ end of the probe or at the 5′ end of another, different, probe part is complementary to the same said nucleotide, but is prevented from hybridizing thereto by a 3′ ligatable end (e.g., it is a displaced nucleotide). Cleavage of the probe to remove the additional sequence provides a 5′ ligatable end, which may be ligated to the 3′ ligatable end of the probe or probe part if the 3′ ligatable end is hybridized correctly to (e.g. is complementary to) the target nucleic acid molecule. Probes designed according to this principle provide a high degree of discrimination between different variants at the position of interest, as only probes in which the 3′ ligatable end is complementary to the nucleotide at the position of interest may participate in a ligation reaction. In one embodiment, the probe is provided in a single part, and the 3′ and 5′ ligatable ends are provided by the same probe. In some embodiments, an invader probe is a padlock probe (an invader padlock or “iLock”), e.g., as described in Krzywkowski et al., Nucleic Acids Research 45, e161, 2017 and US 2020/0224244, which are incorporated herein by reference.

Other types of probes which result in circular molecules which can be detected by RCA and which comprise either a target analyte sequence or a complement thereof include selector-type probes described in US20190144940, which comprise sequences capable of directing the cleavage of a target nucleic acid molecule (e.g. a target analyte) so as to release a fragment comprising a target sequence from the target analyte and sequences capable of templating the circularization and ligation of the fragment. US2018/0327818, which is incorporated herein by reference in its entirety, describes probes which comprise a 3′ sequence capable of hybridizing to a target nucleic acid molecule (e.g. a target analyte) and acting as a primer for the production of a complement of a target sequence within the target nucleic acid molecule (e.g. by target templated extension of the primer), and an internal sequence capable of templating the circularization and ligation of the extended probe comprising the reverse complement of the target sequence within the target analyte and a portion of the probe. In the case of both such probes, target sequences or complements thereof are incorporated into a circularized molecule which acts as the template for the RCA reaction to generate the RCA product, which consequently comprises concatenated repeats of said target sequence. In some embodiments, said target sequence may act as, or may comprise a marker sequence within the RCA product indicative of the target analyte in question. Alternatively, a marker sequence (e.g., tag or barcode sequence) may be provided in the non-target complementary parts of the probes.

In some embodiments, a nucleic acid probe disclosed herein can be pre-assembled from multiple components, e.g., prior to contacting the nucleic acid probe with a target nucleic acid or a sample. In some embodiments, a nucleic acid probe disclosed herein can be assembled during and/or after contacting a target nucleic acid or a sample with multiple components. In some embodiments, a nucleic acid probe disclosed herein is assembled in situ in a sample. In some embodiments, the multiple components can be contacted with a target nucleic acid or a sample in any suitable order and any suitable combination. For instance, a first component and a second component can be contacted with a target nucleic acid, to allow binding between the components and/or binding between the first and/or second components with the target nucleic acid. Optionally a reaction involving either or both components and/or the target nucleic acid, between the components, and/or between either one or both components and the target nucleic acid can be performed, such as hybridization, ligation, primer extension and/or amplification, chemical or enzymatic cleavage, click chemistry, or any combination thereof. In some embodiments, a third component can be added prior to, during, or after the reaction. In some embodiments, a third component can be added prior to, during, or after contacting the sample with the first and/or second components. In some embodiments, the first, second, and third components can be contacted with the sample in any suitable combination, sequentially or simultaneously. In some embodiments, the nucleic acid probe can be assembled in situ in a stepwise manner, each step with the addition of one or more components, or in a dynamic process where all components are assembled together. One or more removing steps, e.g., by washing the sample such as under stringent conditions, may be performed at any point during the assembling process to remove or destabilize undesired intermediates and/or components at that point and increase the chance of accurate probe assembly and specific target binding of the assembled probe.

(ii) Photoactivatable Functional Moieties

The methods and compositions provided herein may comprise an oligonucleotide comprising a photoactivatable functional moiety. In some embodiments, the oligonucleotide comprises one or more photoactivatable functional moieties. In some embodiments, the oligonucleotide comprises more than one photoactivatable functional moiety. In some embodiments, the photoactivatable functional moiety is a diazirine moiety, an aryl azide moiety, a benzophenone moiety, or an anthraquinone moiety. In some embodiments, the photoactivatable functional moiety is a carbene forming moiety.

In some embodiments, the linker is a branched linker, such as any of the branched linkers described herein, comprising a plurality of arms each coupled to one or more of the photoactivatable functional moieties. In some embodiments, the linker may comprise additional chemical groups to increase linearity away from the hybridization region. In some embodiments, the linker comprises an alkene. In some embodiments, the primer comprises at least about 3, at least about 5, at least about 10, at least about 15, at least about 20, or more than 20 of the photoactivatable functional moiety.

In some embodiments, the method comprises reacting the photoactivatable functional moiety with the endogenous molecule prior to, during, or after generating a product of the oligonucleotide (e.g., an amplification) in the biological sample. In some embodiments, the method comprises reacting the photoactivatable functional moiety with the endogenous molecule prior to generating the RCP in the biological sample. In some embodiments, the method comprises reacting the photoactivatable functional moiety with the endogenous molecule during the generating the RCP in the biological sample. In some embodiments, the method comprises reacting the photoactivatable functional moiety with the endogenous molecule after generating the RCP in the biological sample. In some embodiments, reacting the photoactivatable functional moiety with the endogenous molecule immobilizes the RCP in the biological sample.

In some embodiments, the photoactivatable functional moiety is activated by exposing the biological sample to UV light. In some embodiments, the biological sample is exposed to a wavelength of light between about 100 nm and about 450 nm, such as between about 350 nm and about 450 nm. In some embodiments, upon exposure to UV light, the activated photoactivatable functional moiety reacts with the endogenous molecule in the biological sample. In some embodiments, the endogenous molecule is a polypeptide and the activated photoactivatable functional moiety reacts with an amino acid side chain or a peptide backbone of the polypeptide.

### III. Samples, Analytes, and Target Sequences

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, a section of a cell block or cell pellet, a biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

(i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

(ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

(iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

(v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Methods of destaining or discoloring a biological sample generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

(vi) Isometric Expansion

In some embodiments, a biological sample embedded in a matrix (e.g., a hydrogel) can be isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of analytes out of the sample, and/or to facilitate transfer of species (such as probes) into the sample. If a sample is not permeabilized sufficiently, the amount of analyte captured from the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, an oligonucleotide with sequence complementarity to the complementary strand of RNA (e.g., cDNA) can bind to the cDNA. For example, biotinylated oligonucleotides with sequence complementary to one or more cDNA of interest binds to the cDNA and can be selected using biotinylation-strepavidin affinity using any of a variety of methods (e.g., streptavidin beads).

Alternatively, one or more species of RNA can be down-selected (e.g., removed) using any of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

A biological sample may comprise one or a plurality of analytes of interest. Methods for performing multiplexed assays to analyze two or more different analytes in a single biological sample are provided.

B. Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus, in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

(i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

In any embodiment described herein, the analyte comprises a target sequence. In some embodiments, the target sequence may be endogenous to the sample, generated in the sample, added to the sample, or associated with an analyte in the sample. In some embodiments, the target sequence is a single-stranded target sequence (e.g., a sequence in a rolling circle amplification product). In some embodiments, the analytes comprise one or more single-stranded target sequences. In one aspect, a first single-stranded target sequence is not identical to a second single-stranded target sequence. In another aspect, a first single-stranded target sequence is identical to one or more second single-stranded target sequence. In some embodiments, the one or more second single-stranded target sequence is comprised in the same analyte (e.g., nucleic acid) as the first single-stranded target sequence. Alternatively, the one or more second single-stranded target sequence is comprised in a different analyte (e.g., nucleic acid) from the first single-stranded target sequence.

(ii) Labeling Agents

In some embodiments, provided herein are methods and compositions for analyzing endogenous analytes (e.g., RNA, ssDNA, and cell surface or intracellular proteins and/or metabolites) in a sample using one or more labeling agents. In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or antigen binding fragments thereof. The antibodies or antigen binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labeling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed. In some embodiments, an endogenous analyte (e.g., a viral or cellular DNA or RNA) or a product thereof is analyzed using a probe comprising: i) a hybridization region complementary to a target nucleic acid sequence, ii) a plurality of detectable labels, and iii) a branched linker coupled to the hybridization region, as described in Section IIB. In some embodiments, an endogenous analyte (e.g., a viral or cellular DNA or RNA) or a product thereof is contacted with an oligonucleotide comprising: i) a hybridization region complementary to a target nucleic acid sequence, ii) a photoactivatable functional moiety capable of covalently bonding to an endogenous molecule in the biological sample, and iii) a linker coupling the functional moiety to the hybridization region, to immobilize the oligonucleotide in the biological sample, as described in Section II.C.

(a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a padlock probe, a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary.

(b) Ligation

In some embodiments, a product of an endogenous analyte and/or a labeling agent is a ligation product. In some embodiments, the ligation product is formed between two or more endogenous analytes. In some embodiments, the ligation product is formed between an endogenous analyte and a labeling agent. In some embodiments, the ligation product is formed between two or more labeling agent. In some embodiments, the ligation product is an intramolecular ligation of an endogenous analyte. In some embodiments, the ligation product is an intramolecular ligation of a labeling agent, for example, the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, provided herein is a probe or probe set capable of DNA-templated ligation, such as from a cDNA molecule. See, e.g., U.S. Pat. No. 8,551,710, which is hereby incorporated by reference in its entirety. In some embodiments, provided herein is a probe or probe set capable of RNA-templated ligation. See, e.g., U.S. Pat. Pub. 2020/0224244 which is hereby incorporated by reference in its entirety. In some embodiments, the probe set is a SNAIL probe set. See, e.g., U.S. Pat. Pub. 20190055594, which is hereby incorporated by reference in its entirety. In some embodiments, provided herein is a multiplexed proximity ligation assay. See, e.g., U.S. Pat. Pub. 20140194311 which is hereby incorporated by reference in its entirety. In some embodiments, provided herein is a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, a circular probe can be indirectly hybridized to the target nucleic acid. In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation. In some embodiments, the ligation involves template dependent ligation. In some embodiments, the ligation involves template independent ligation. In some embodiments, the ligation involves enzymatic ligation.

In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, padlock probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

(c) Primer Extension and Amplification

In some embodiments, a product is a primer extension product of an analyte, a labeling agent, a probe or probe set bound to the analyte (e.g., a padlock probe bound to genomic DNA, mRNA, or cDNA), or a probe or probe set bound to the labeling agent (e.g., a padlock probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., for example, 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, a product of an endogenous analyte and/or a labeling agent is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (e.g., amplicon) containing multiple copies of the cDNA. Techniques for rolling circle amplification (RCA) can include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, US 2022/0228196, US 2021/0363579, US 2016/0024555, US 2018/0251833 and US 2017/0219465. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress. In some embodiments, the amplification products can be immobilized using methods involving photoactivatable functional moieties as described in Section II.C.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, assays for the detection of numerous different analytes can use a RCA-based detection system, e.g., where the signal is provided by generating a RCP from a circular RCA template which is provided or generated in the assay, and the RCP is detected to detect the analyte. The RCP may thus be regarded as a reporter which is detected to detect the target analyte. However, the RCA template may also be regarded as a reporter for the target analyte; the RCP is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g. a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

In some embodiments, a product herein includes a molecule or a complex generated in a series of reactions, e.g., hybridization, ligation, extension, replication, transcription/reverse transcription, and/or amplification (e.g., rolling circle amplification), in any suitable combination. For example, a product comprising a target sequence for a probe disclosed herein may be a hybridization complex formed of a cellular nucleic acid in a sample and an exogenously added nucleic acid probe. The exogenously added nucleic acid probe may comprise an overhang that does not hybridize to the cellular nucleic acid but hybridizes to another probe. The exogenously added nucleic acid probe may be optionally ligated to a cellular nucleic acid molecule or another exogenous nucleic acid molecule. In other examples, a product comprising a target sequence for a probe disclosed herein may be an RCP of a circularizable probe or probe set which hybridizes to a cellular nucleic acid molecule (e.g., genomic DNA or mRNA) or product thereof (e.g., a transcript such as cDNA, a DNA-templated ligation product of two probes, or an RNA-templated ligation product of two probes). In other examples, a product comprising a target sequence for a probe disclosed herein may a probe hybridizing to an RCP. The probe may comprise an overhang that does not hybridize to the RCP but hybridizes to another probe. The probe may be optionally ligated to a cellular nucleic acid molecule or another probe that hybridizes to the RCP.

C. Target Sequences

A target sequence for a probe or oligonucleotide disclosed herein may be comprised in any analyte disclose herein, including an endogenous analyte (e.g., a viral or cellular nucleic acid), a labeling agent, or a product of an endogenous analyte and/or a labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”). In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In any of the preceding embodiments, barcodes can be analyzed (e.g., detected or sequenced) using any suitable methods or techniques, including those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), in situ sequencing, hybridization-based in situ sequencing (HybISS), targeted in situ sequencing, fluorescent in situ sequencing (FISSEQ), sequencing by synthesis (SBS), sequencing by ligation (SBL), sequencing by hybridization (SBH), or spatially-resolved transcript amplicon readout mapping (STARmap). In any of the preceding embodiments, the methods provided herein can include analyzing the barcodes by sequential hybridization and detection with a plurality of labeled probes (e.g., detection oligos). In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., US 2019/0055594 A1 and US 2021/0164039 A1, which are hereby incorporated by reference in their entirety.

### IV. Detection and Analysis

In some aspects, the provided methods involve analyzing, e.g., detecting or determining, one or more target sequences present in a target molecule described herein. In some embodiments, a target nucleic acid is present in the target molecules (e.g., nucleic acid concatemers, such as RCPs) described herein. In some embodiments, the detecting comprises hybridizing one or more detectably labeled probes to a nucleic acid concatemer, or via hybridization to intermediate probes that hybridize to the nucleic acid concatemer). In some embodiments, the analysis comprises determining the sequence of all or a portion of the nucleic acid concatemer (e.g., a barcode sequence or a complement thereof), wherein the sequence is indicative of a sequence of the target nucleic acid.

In some cases, analysis is performed on one or more images captured, and may comprise processing the image(s) and/or quantifying signals observed. In some embodiments, images of signals from different fluorescent channels and/or detectable probe hybridization cycles can be compared and analyzed. In some embodiments, images of signals (or absence thereof) at a particular location in a sample from different fluorescent channels and/or sequential detectable probe hybridization cycles can be aligned to analyze an analyte at the location. For instance, a particular location in a sample can be tracked and signal spots from sequential probe hybridization (and optionally ligation) cycles can be analyzed to detect a target polynucleotide sequence (e.g., a barcode sequence or subsequence thereof) in a nucleic acid at the location. The analysis may comprise processing information of one or more cell types, one or more types of analytes, a number or level of analyte, and/or a number or level of cells detected in a particular region of the sample. In some embodiments, the analysis comprises detecting a sequence e.g., a barcode sequence present in an amplification product at a location in the sample. In some embodiments, the analysis includes quantification of puncta (e.g., if amplification products are detected). In some cases, the analysis includes determining whether particular cells and/or signals are present that correlate with one or more analytes from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection step. In some embodiments, software may be used to automate the processing, analysis, and/or display of data.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization in a FISH-type assay, sequencing by hybridization).

In some embodiments, the methods comprise sequencing all or a portion of a target molecule (e.g., nucleic acid concatemer, RCP), such as one or more barcode sequences present in the nucleic acid concatemer. In some embodiments, the sequence of the nucleic acid concatemer, or barcode thereof, is indicative of a sequence of the target nucleic acid to which the nucleic acid concatemer is hybridized. In some embodiments, the analysis and/or sequence determination comprises sequencing all or a portion of the nucleic acid concatemer and/or in situ hybridization to the nucleic acid concatemer. In some embodiments, the sequencing step involves sequencing by hybridization, sequencing by ligation, sequencing by synthesis, sequencing by binding, and/or fluorescent in situ sequencing (FISSEQ), hybridization-based in situ sequencing and/or wherein the in situ hybridization comprises sequential fluorescent in situ hybridization. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product/nucleic acid concatemer).

In some aspects, the provided methods comprise imaging the probe hybridized to the target molecule (e.g., nucleic acid concatemer, RCP), for example, when the probe is a detectably labeled probe and the method comprises detecting the detectable label (e.g., probes comprising branched linkers, such as the probes described in Section IIB). In some embodiments, the probe comprises one or more detectable labels that can be measured and quantitated. The terms “label” and “detectable label” comprise a directly or indirectly detectable moiety that is associated with (e.g., conjugated to) a molecule to be detected, e.g., a probe that is a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, sequence analysis of nucleic acids (e.g., nucleic acids such as probes or RCPs comprising barcode sequences generated using the circular nucleic acid as template) can be performed by sequential hybridization (e.g., sequencing by hybridization and/or sequential in situ fluorescence hybridization). Sequential fluorescence hybridization can involve sequential hybridization of detectable probes comprising an oligonucleotide and a detectable label. In some embodiments, a method disclosed herein comprises sequential hybridization of the detectable probes disclosed herein, including detectably labeled probes (e.g., fluorophore conjugated oligonucleotides) and/or probes that are not detectably labeled per se but are capable of binding (e.g., via nucleic acid hybridization) and being detected by detectably labeled probes. Exemplary methods comprising sequential fluorescence hybridization of detectable probes are described in US 2019/0161796, US 2020/0224244, US 2022/0010358, US 2021/0340618, and US 2023/0039899, all of which are incorporated herein by reference. In some embodiments, the methods provided herein can include analyzing the identifier sequences (e.g., analyte sequences or barcode sequences) by sequential hybridization and detection with a plurality of labeled probes (e.g., detection oligonucleotides).

In some embodiments, sequence detection comprises contacting the biological sample with one or more intermediate probes that directly or indirectly hybridize to the RCP, wherein the one or more intermediate probes are detectable using one or more detectably-labeled probes (e.g., probes comprising branched linkers, such as the probes described in Section IIB), and dehybridizing the one or more intermediate probes and/or the one or more detectably-labeled probes from the rolling circle amplification product. In some aspects, dehybridizing probes comprises removal of the probes from the RCP. In some embodiments, the one or more intermediate probes comprise one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the biological sample with a pool of intermediate probes corresponding to different barcode sequences or portions thereof, and a pool of detectably-labeled probes corresponding to different detectable labels. In some embodiments, the biological sample is sequentially contacted with different pools of intermediate probes. In some instances, a common or universal pool of detectably-labeled probes is used in a plurality of sequential hybridization steps (e.g., with different pools of intermediate probes).

In some embodiments, provided herein are methods for in situ analysis of analytes in a sample using sequential probe hybridization. In some aspects provided herein is a method for analyzing a biological sample, comprising: a) generating a rolling circle amplification product (RCP) of a circular probe or circularizable probe or probe set described herein, the RCP comprising an identifier sequence such as a barcode sequence or analyte sequence, wherein the identifier sequence is associated with an analyte of interest and is assigned a signal code sequence; b) contacting the biological sample with a first probe (e.g., an intermediate probe such as an L-probe) and a first detectably labeled probe to generate a first complex comprising the first probe hybridized to the RCP and the first detectably labeled probe hybridized to the first probe, wherein the first probe comprises (i) a recognition sequence (e.g., a target-binding sequence) complementary to the identifier sequence (e.g., barcode sequence or analyte sequence) and (ii) a first landing sequence (e.g., an overhang sequence), and wherein the first detectably labeled probe comprises a sequence complementary to the first landing sequence; c) detecting a first signal associated with the first detectably labeled probe, wherein the first signal corresponds to a first signal code in the signal code sequence; d) contacting the biological sample with a second probe (e.g., an intermediate probe such as L-probe) and a second detectably labeled probe to generate a second complex comprising the second probe hybridized to the RCP and the second detectably labeled probe hybridized to the second probe, wherein the second probe comprises (i) a recognition sequence (e.g., a target-binding sequence) complementary to the identifier sequence (e.g., barcode sequence or analyte sequence) and (ii) a second landing sequence (e.g., an overhang sequence), and wherein the second detectably labeled probe comprises a sequence complementary to the second landing sequence; and e) detecting a second signal associated with the second detectably labeled probe, wherein the second signal corresponds to a second signal code in the signal code sequence, wherein the signal code sequence comprising the first signal code and the second signal code is determined at a location in the biological sample, thereby decoding the identifier sequence (e.g., barcode sequence or analyte sequence) and identifying the analyte of interest at the location in the biological sample. In some embodiments, the detectable label of the first detectably labeled probe and the detectable label of the second detectably labeled probe are the same. In some embodiments, the detectable labels of the first detectably labeled probe and the second detectably labeled probe are different. In some embodiments, the first signal code and the second signal code are the same. In some embodiments, the first signal code and the second signal code are different.

In some embodiments, the first probe (e.g., a first intermediate probe such as a first L-probe), the second probe (e.g., a second intermediate probe such as a second L-probe), and one or more subsequent probes (e.g., subsequent intermediate probe such as subsequent L-probes) are contacted with the biological sample sequentially in a pre-determined sequence which corresponds to the signal code sequence assigned to the identifier sequence (e.g., barcode sequence or analyte sequence), wherein the one or more subsequent probes each comprises (i) a recognition sequence complementary to the identifier sequence (e.g., barcode sequence or analyte sequence) and (ii) an overhang sequence complementary to a detectably labeled probe of a pool (e.g., a universal pool across different cycles of probe hybridization) of detectably labeled probes. In some embodiments, the biological sample is contacted with the first probe before the second probe and one or more subsequent probes. In some embodiments, the biological sample is contacted with the second after the first probe and before and one or more subsequent probes. In some embodiments, the biological sample is contacted with the one or more subsequent probes after the first probe. In some embodiments, the biological sample is contacted with the one or more subsequent probes after the first probe and the second probe.

In some embodiments, the first detectably labeled probe and the second detectably labeled probe are in the pool of detectably labeled probes. A pool of detectably labeled probes may comprise at least two detectably labeled probes, and may be used for multiplexing analyses of two or more target analytes (e.g., target nucleic acids) in a biological sample. In some embodiments, the contacting in b) comprises contacting the biological sample with the universal pool of detectably labeled probes, and the contacting in d) comprises contacting the biological sample with the universal pool of detectably labeled probes. In some embodiments, the universal pool of detectably labeled probes used in the contacting in b) is the same as the universal pool of detectably labeled probes used in the contacting in d). In some embodiments, the universal pool comprises detectably labeled probes each having a detectable label corresponding to a different nucleic acid sequence for hybridization to a landing sequence (e.g., an overhang sequence) in a probe (e.g., an intermediate probe such as an L-probe). In some embodiments, the number of different detectably labeled probes in the universal pool is four.

In some embodiments, the one or more subsequent probes are contacted with the biological sample to determine signal codes in the signal code sequence until sufficient signal codes have been determined to decode the identifier sequence (e.g., barcode sequence or analyte sequence), thereby identifying the target analyte (e.g., target nucleic acid). In some embodiments, the method further comprises a step of removing the first probe and/or the first detectably labeled probe from the biological sample before contacting the sample with a subsequent probe and a detectably labeled probe hybridizing to the subsequent probe. In some embodiments, the method further comprises a step of removing the second probe and/or the second detectably labeled probe from the biological sample, before contacting the sample with a subsequent probe and a detectably labeled probe hybridizing to the subsequent probe.

In some embodiments, the method further comprises identifying multiple different target analytes present at locations (e.g., different locations) in the biological sample. In some embodiments, each different target analyte is assigned a different signal code sequence and is targeted by a circular or circularizable probe or probe set comprising a complement of a different barcode sequence of the plurality of barcode sequences. In some embodiments, the number of different probes (e.g., L-probes that have different recognition sequences that bind to the barcode sequences) in each pool of probes is greater than the number of different detectably labeled probes in the universal pool of detectably labeled probes. In some embodiments, the number of different detectably labeled probes in the universal pool is four. In some embodiments, the number of different probes in each pool of probes (e.g., L-probes) is about 10, about 20, about 30, about 40, about 50, about 100, about 200, about 500, about 1,000, or more. In some embodiments, the number of different recognition sequences (e.g., recognition sequences that bind to the barcode sequences) of probes in each pool of probes in at least about 10, such as at least any of about 20, 30, 40, 50, 100, 200, 500, 1,000, or more.

In some embodiments, a method disclosed herein may also comprise one or more signal amplification components. In some embodiments, the present disclosure relates to the detection of nucleic acids sequences in situ using probe hybridization and generation of amplified signals associated with the probes, wherein background signal is reduced and sensitivity is increased. In some embodiments, the RCA product can be detected in with a method that comprises signal amplification.

Exemplary signal amplification methods include targeted deposition of detectable reactive molecules around the site of probe hybridization, targeted assembly of branched structures (e.g., bDNA or branched assay using locked nucleic acid (LNA)) or programmed in situ growth of concatemers by enzymatic rolling circle amplification (RCA) (e.g., as described in US 2019/0055594 incorporated herein by reference). In some embodiments, detection of nucleic acids sequences in situ includes an assembly for branched signal amplification. In some embodiments, the assembly complex comprises an amplifier hybridized directly or indirectly (via one or more oligonucleotides) to a sequence of a probe hybridized to a target nucleic acid or a product thereof. In some embodiments, the assembly includes one or more amplifiers each including an amplifier repeating sequence.

In some embodiments, the RCP can be detected by providing detection probes (e.g., detection probes comprising branched linkers, such as the probes described in Section IIB), such as probes for performing a chain reaction that forms an amplification product, e.g., HCR. In some embodiments, the analysis comprises determining the sequence of all or a portion of the amplification product. In some embodiments, the analysis comprises detecting a sequence present in the amplification product. In some embodiments, the sequence of all or a portion of the amplification product is indicative of the identity of a region of interest in a target nucleic acid. In other embodiments, the provided methods involve analyzing, e.g., detecting or determining, one or more sequences present in the polynucleotide probes.

The term “fluorophore” comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some embodiments, a detectable probe containing a detectable label (e.g., probes comprising branched linkers, such as the probes described in Section IIB) can be used to detect one or more target molecule, such as nucleic acid concatemer(s), described herein. In some embodiments, the methods involve incubating the detectable probe containing the detectable label with the sample, washing unbound detectable probe, and detecting the label, e.g., by imaging. In some embodiments, the nucleic acid concatemer(s) remain crosslinked to the target nucleic acid during the washing and detecting steps.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264. In some embodiments, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-!2-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods for custom synthesis of nucleotides having other fluorophores include those described in Henegariu et al. (2000) Nature Biotechnol. 18:345.

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In some embodiments, the term antibody refers to an antibody molecule of any class, or any sub-fragment thereof, such as an Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an oligonucleotide sequence can be indirectly labeled, especially with a hapten that is then bound by a capture agent, e.g., as disclosed in U.S. Pat. Nos. 5,344,757, 5,702,888, 5,354,657, 5,198,537 and 4,849,336, and PCT publication WO 91/17160. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe (e.g., probes comprising branched linkers, such as the probes described in Section II.B). In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe (e.g., probes comprising branched linkers, such as the probes described in Section II.B). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequence determination can be performed in situ. In situ sequence determination typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121).

In some embodiments, sequence determination can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232.

In some embodiments, sequence determination can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597.

In some embodiments, the barcodes of the detection probes are targeted by detectably labeled oligonucleotides, such as fluorescently labeled oligonucleotides. In some embodiments, one or more decoding schemes are used to decode the signals, such as fluorescence, for sequence determination. In any of the embodiments herein, barcodes can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labeled probes (e.g., detection oligonucleotides). Exemplary decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233):aaa6090 (2015); Gyllborg et al., Nucleic Acids Res (2020) 48(19):e112; U.S. Pat. No. 10,457,980 B2; US 2016/0369329 A1; WO 2018/026873 A1; US 2020/0080139 A1; US 2021/0017587 A1; and US 2017/0220733 A1, all of which are incorporated by reference in their entirety. In some embodiments, these assays enable signal amplification, combinatorial decoding, and error correction schemes at the same time.

In some embodiments, nucleic acid hybridization can be used for sequence determination. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004).

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### V. Compositions, Kits, and Systems

In some embodiments, disclosed herein is a probe comprising: i) a hybridization region complementary to a target nucleic acid sequence (e.g., a target nucleic amplification product, such as a nucleic acid concatemer, e.g., a RCP), ii) a plurality of detectable labels, and iii) a branched linker coupled to the hybridization region, wherein the branched linker comprises a plurality of arms each coupled to one or more detectable labels, e.g., any of the target nucleic acid sequences and probes described in Section II.B. In some aspects, provided herein is a composition that comprises a complex containing a target nucleic acid (e.g., a target nucleic amplification product, such as a nucleic acid concatemer, e.g., a RCP) and a probe comprising: i) a hybridization region complementary to the target nucleic acid sequence, ii) a plurality of detectable labels, and iii) a branched linker coupled to the hybridization region, wherein the branched linker comprises a plurality of arms each coupled to one or more detectable labels, wherein the hybridization region of the probe is hybridized to the target nucleic acid sequence e.g., any of the target nucleic acid sequences, detectable labels, and probes described in Section IIB. By configuring the probes to comprise a plurality of detectable labels using branched linkers, the signal associated with the detectable label may be stronger, thus improving the detection of the target nucleic acid sequence.

In some embodiments, disclosed herein is an oligonucleotide comprising: i) a hybridization region complementary to a target nucleic acid sequence, ii) one or more photoactivatable functional moieties, and iii) a branched linker coupled to the hybridization region, wherein the branched linker comprises a plurality of arms each coupled to the one or more photoactivatable functional moieties, e.g., any of the target nucleic acid sequences, oligonucleotides, and photoactivatable functional moieties described in Section II.C. In some aspects, provided herein is a composition that comprises a complex containing a target nucleic acid (e.g., a target nucleic amplification product, such as a nucleic acid concatemer, e.g., a RCP) and an oligonucleotide comprising: i) a hybridization region complementary to the target nucleic acid sequence, ii) one or more photoactivatable functional moieties, and iii) a branched linker coupled to the hybridization region, wherein the branched linker comprises a plurality of arms each coupled to the one or more photoactivatable functional moieties, wherein the hybridization region of the oligonucleotide is hybridized to the target nucleic acid sequence e.g., any of the target nucleic acid sequences, oligonucleotides, and photoactivatable functional moieties described in Section II.C. By configuring the oligonucleotide to comprise one or more photoactivatable functional moieties using branched linkers, the target nucleic acid sequence, or product thereof, may be tethered at a location in a biological sample, thus immobilizing the target nucleic acid sequence or product thereof in the biological sample.

Also provided herein are kits, for example comprising one or more probes and/or oligonucleotide, e.g., any described in Section II, and instructions for performing the methods provided herein. In some embodiments, the kits further comprise one or more reagents required for one or more steps comprising hybridization, ligation, extension, detection, sequencing, and/or sample preparation as described herein. In some embodiments, the kit further comprises a target nucleic acid, e.g., any described in Sections II and III. In some embodiments, any or all of the probes and/or oligonucleotides are DNA molecules. In some embodiments, the target nucleic acid is a messenger RNA molecule. In some embodiments, the target nucleic acid is a probe (e.g., a padlock probe, such as a molecule for generating a nucleic acid concatemer) or an amplification product thereof (e.g., a RCP, such as a nucleic acid concatemer). The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers. In some aspects, the kit can also comprise any of the reagents described herein, e.g., wash buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, and reagents for additional assays.

In some embodiments, provided herein is a system comprising the probes or oligonucleotides provided herein and a source (e.g., providing UV light) for activating a photoactivatable functional moiety in a biological sample. In some embodiments, the system further comprises one or more computer processors operatively coupled to the source (e.g., providing UV light) for activating a photoactivatable functional moiety.

### VI. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis. In some aspects, the provided embodiments can be used to immobilize a nucleic acid concatemer in a space in a biological sample via a photoactivatable functional moiety that may be reacted with an endogenous molecule in the biological sample, to increase the resolution and stability of the nucleic acid concatemers in situ.

In some embodiments, a region of interest for the analytical methods provided herein comprises a single-nucleotide polymorphism (SNP). In some embodiments, the region of interest comprises is a single-nucleotide variant (SNV). In some embodiments, the region of interest comprises a single-nucleotide substitution. In some embodiments, the region of interest comprises a point mutation. In some embodiments, the region of interest comprises a single-nucleotide insertion.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VII. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. An estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one probe, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

As used herein, the term “alkyl”, by itself or as part of another substituent, means, unless otherwise stated, a straight or branched chain hydrocarbon radical, having the number of carbon atoms designated (e.g., C₁-C₆ means one to six carbons). Examples of alkyl groups include methyl, ethyl, n-propyl, iso-propyl, n-butyl, t-butyl, iso-butyl, sec-butyl, n-pentyl, n-hexyl, n-heptyl, n-octyl, and the like. In some embodiments, the term “alkyl” may encompass C₁-C₆ alkyl, C₂-C₆ alkyl, C₃-C₆ alkyl, C₄-C₆ alkyl, C₅-C₆ alkyl, C₁-C₅ alkyl, C₂-C₅ alkyl, C₃-C₅ alkyl, C₄-C₅ alkyl, C₁-C₄ alkyl, C₂-C₄ alkyl, C₃-C₄ alkyl, C₁-C₃ alkyl, C₂-C₃ alkyl, or C₁-C₂ alkyl.

As used herein, the term “alkenyl”, by itself or as part of another substituent, means, unless otherwise stated, a straight or branched chain hydrocarbon radical having one or more double bond functional groups (e.g., a —CH═CH— moiety), having the number of carbon atoms designated (e.g., C₂-C₆ means two to six carbons). An alkenyl group may comprise 1, 2, 3, 4, 5 or 6 or more double bond functional groups (e.g., a —CH═CH— moiety). An alkenyl group having multiple double bonds may have the double bonds in conjugation (e.g., a 1,3-butadienyl group) or not in conjugation (e.g., with one or more intervening saturated carbon atoms). Examples of alkenyl groups include ethenyl (e.g., vinyl), propenyl, allyl, isopropenyl, butenyl, pentenyl, hexenyl, and the like.

As used herein, the term “alkynyl”, by itself or as part of another substituent, means, unless otherwise stated, a straight or branched chain hydrocarbon radical having one or more triple bond functional groups (e.g., a —C≡C— moiety), having the number of carbon atoms designated (e.g., C₂-C₆ means two to six carbons). An alkynyl group may comprise 1, 2, 3, 4, 5 or 6 or more double bond functional groups (e.g., a —C≡C— moiety). An alkynyl group having multiple triple bonds may have the triple bonds in conjugation (e.g., a polyacetylene) or not in conjugation (e.g., with one or more intervening saturated carbon atoms). Examples of alkenyl groups include ethynyl, propynyl, butynyl, pentynyl, hexynyl, and the like.

As used herein, “poly(ethylene glycol)” (e.g., “PEG”, “polyethylene glycol”, or “polyethyleneglycol”) refers to a polymer or polymer moiety having a repetition of ethylene glycol units of the formula

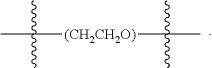

PEGs include polydisperse PEGs and monodisperse PEGs. Polydisperse PEGs are characterized by a distribution of sizes and molecular weights, whereas monodisperse PEGs are typically purified polymers or polymer moieties of a single chain length and molecular weight.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-72. (canceled)

73. A method for analyzing a biological sample, comprising:
a) contacting the biological sample with an oligonucleotide comprising:
i) a hybridization region complementary to a target nucleic acid sequence in a target molecule,
ii) a photoactivatable functional moiety capable of covalently bonding to an endogenous molecule in the biological sample, and
iii) a linker coupling the functional moiety to the hybridization region,
thereby hybridizing the oligonucleotide to the target molecule at a location in the biological sample;
b) photoactivating the photoactivatable functional moiety to react with the endogenous molecule, thereby immobilizing the oligonucleotide in the biological sample;
c) contacting the biological sample with a detectable probe that directly or indirectly binds to the oligonucleotide or a product thereof; and
d) detecting a signal associated with the detectable probe at the location in the biological sample.

74. The method of claim 73, wherein the target molecule is a genomic DNA, mRNA, or cDNA, or a product thereof.

75. The method of claim 73, wherein the target molecule is a nucleic acid probe that hybridizes to a genomic DNA, mRNA, or cDNA.

76. The method of claim 73, wherein the target molecule is a nucleic acid probe that hybridizes to a genomic DNA, mRNA, or cDNA.

77. The method of claim 73, wherein the target molecule is a circular or circularizable probe or probe set, the oligonucleotide primes rolling circle amplification of the circular probe or a circularized probe generated from the circularizable probe or probe set, and the product of the oligonucleotide is a rolling circle amplification product (RCP).

78. The method of claim 73, wherein the endogenous molecule is a protein, a nucleic acid, a carbohydrate, or a lipid.

79. The method of claim 73, wherein the photoactivatable functional moiety is a diazirine moiety, an aryl azide moiety, a benzophenone moiety, or an anthraquinone moiety.

80. The method of claim 73, wherein the photoactivatable functional moiety is a carbene forming moiety.

81. The method of claim 73, wherein the linker comprises a plurality of arms each coupled to one or more copies of the photoactivatable functional moiety.

82. The method of claim 73, wherein the linker comprises an alkene.

83. The method of claim 73, wherein the linker comprises a double branch point linker, a triple branch point linker, and/or a linker with more than three branch points.

84. The method of claim 73, wherein the linker comprises a phosphoramidite.

85. (canceled)

86. The method of claim 77, comprising photoactivating the photoactivatable functional moiety to react with the endogenous molecule prior to generating the product of the oligonucleotide in the biological sample, thereby immobilizing the product in the biological sample.

87. The method of claim 86, wherein the photoactivatable functional moiety is photoactivated by exposing the biological sample to UV light, and the activated photoactivatable functional moiety reacts with the endogenous molecule.

88. The method of claim 73, wherein the linker comprises or is generated using any one or more of:

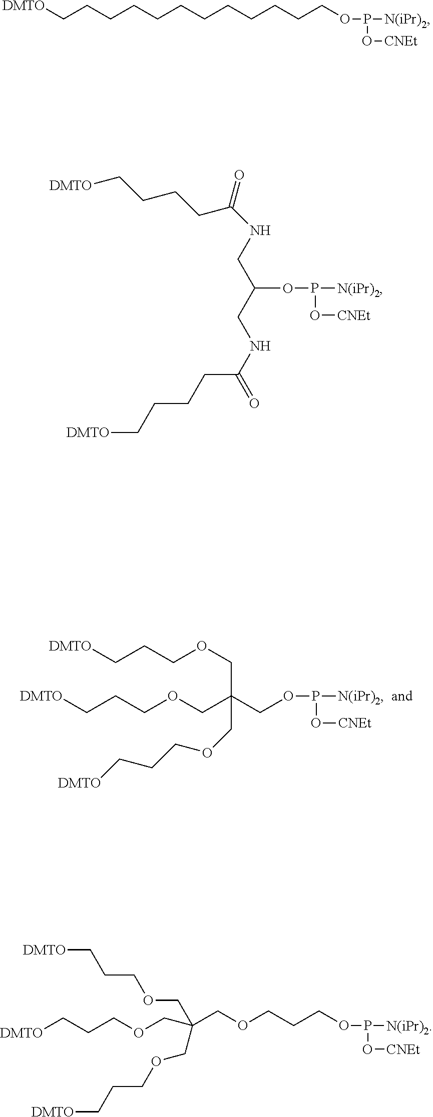

89. (canceled)

90. The method of claim 73, wherein the linker comprises or is generated using any one or more of:

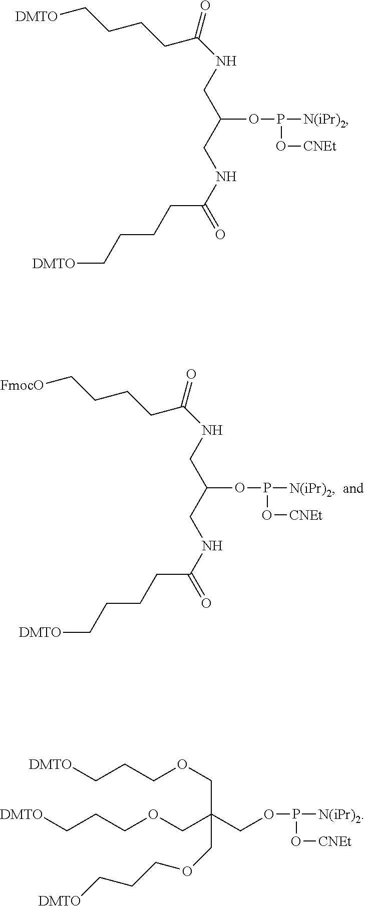

91. The method of claim 81, wherein the plurality of arms each coupled to one or more copies of the photoactivatable functional moiety are reacted with two or more different endogenous molecules in the biological sample.

92. A method for analyzing a biological sample, comprising:
a) hybridizing the oligonucleotide to the target molecule at a location in the biological sample, wherein the oligonucleotide comprises:
i) a hybridization region complementary to a target nucleic acid sequence in a target molecule, wherein the target molecule is a circular or circularizable probe or probe set, the oligonucleotide primes rolling circle amplification of the circular probe or a circularized probe generated from the circularizable probe or probe set, and the product of the oligonucleotide is a rolling circle amplification product (RCP),
ii) a photoactivatable functional moiety capable of covalently bonding to an endogenous molecule in the biological sample, and
iii) a branched linker comprising a plurality of arms, and one or more of the arms are coupled to one or more functional moieties;
b) photoactivating the photoactivatable functional moiety to react with the endogenous molecule, thereby immobilizing the oligonucleotide in the biological sample;
c) contacting the biological sample with a detectable probe that directly or indirectly binds to the oligonucleotide or a product thereof; and
d) detecting a signal associated with the detectable probe at the location in the biological sample.

93. The method of claim 92, wherein the photoactivatable functional moiety is a diazirine moiety, an aryl azide moiety, a benzophenone moiety, or an anthraquinone moiety.

94. The method of claim 92, wherein the photoactivatable functional moiety is a carbene forming moiety.
